SEQUENCE LISTING (31 sequences)

SEQ ID NO: 1 - protein, 96 aa, human herpesvirus 2
MGLAFSGARPCCCRHNVIITDGGEVVSLTAHEFDVVDIESEEEGNFYVPPDMRVVTRAPGPQYRRASDPPSRHTRRRDPDVARPPATLTPPLSDSE

SEQ ID NO: 2 - protein, 372 aa, human herpesvirus 2
MAQRALWRPQATPGPPGAAAPPGHRGAPPDARAPDPGPEADLVARIANSVFVWRVVRGDERLKIFRCLTVLTEPLCQVALPDPDPERALFCEIFLYLTRPKALRLPSNTFFAIFFFNRERRYCATVHLRSVTHPRTPLLCTLAFGHLEAASPPEETPDPAAEQLADEPVAHELDGAYLVPTEPPPNPGACCALGPGAWWHLPGGRIYCWAMDDDLGSLCPPGSRARHLGWLLSRITDPPGGGGACAPTAHIDSANALWRAPAVAEACPCVAPCMWSNMAQRTLAVRGDASLCQLLFGHPVDAVILRQATRRPRITAHLHEVVVGRDGAESVIRPTSAGWRLCVLSSYTSRLFATSCPAVARAVARASSSDYK

SEQ ID NO: 3 - protein, 532 aa, human herpesvirus 2
MELSYATTLHHRDVVFYVTADRNRAYFVCGGSVYSVGRPRDSQPGEIAKFGLVVRGTGPKDRMVANYVRSELRQRGLRDVRPVGEDEVFLDSVCLLNPNVSSERDVINTNDVEVLDECLAEYCTSLRTSPGVLVTGVRVRARDRVIELFEHPAIVNISSRFAYTPSPYVFALAQAHLPRLPSSLEPLVSGLFDGIPAPRQPLDARDRRTDVVITGTRAPRPMAGTGAGGAGAKRATVSEFVQVKHIDRVVSPSVSSAPPPSAPDASLPPPGLQEAAPPGPPLRELWWVFYAGDRALEEPHAESGLTREEVRAVHGFREQAWKLFGSVGAPRAFLGAALALSPTQKLAVYYYLIHRERRMSPFPALVRLVGRYIQRHGLYVPAPDEPTLADAMNGLFRDALAAGTVAEQLLMFDLLPPKDVPVGSDARADSAALLRFVDSQRLTPGGSVSPEHVMYLGAFLGVLYAGHGRLAAATHTARLTGVTSLVLTVGDVDRMSAFDRGPAGAAGRTRTAGYLDALLTVCLARAQHGQSV

SEQ ID NO: 4 - protein, 548 aa, human herpesvirus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 5 - protein, 106 aa, Artificial Sequence
  Cytoplasmic tail of gE protein of HSV-2 (human herpesvirus 2)
RRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 6 - protein, 490 aa, human herpesvirus 2
MDLLVDDLFADADGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDAMGIDDFGG

SEQ ID NO: 7 - protein, 302 aa, human herpesvirus 2
MTSRRSVKSCPREAPRGTHEELYYGPVSPADPESPRDDFRRGAGPMRARPRGEVRFLHYDEAGYALYRDSSSSEDNDESRDTARPRRSASVAGSHGPGPARAPPPPGGPVGAGGRSHAPPARTPKMTRGAPKAPATPATDPARGRRPAQADSAVLLDAPAPTASGRTKTPAQGLAKKLHFSTAPPSPTAPWTPRVAGFNKRVFCAAVGRLAATHARLAAVQLWDMSRPHTDEDLNELLDLTTIRVTVCEGKNLLQRANELVNPDAAQDVDATAAARGRPAGRAAATARAPARSASRPRRPLE

SEQ ID NO: 8 - protein, 305 aa, human herpesvirus 2
MYDIAPRRSGSRPGPGRDKTRRRSRFSAAGNPGVERRASRKSLPSHARRLELCLHERRRYRGFFAALAQTPSEEIAIVRSLSVPLVKTTPVSLPFSLDQTVADNCLTLSGMGYYLGIGGCCPACSAGDGRLATVSREALILAFVQQINTIFEHRTFLASLVVLADRHSTPLQDLLADTLGQPELFFVHTILRGGGACDPRFLFYPDPTYGGHMLYVIFPGTSAHLHYRLIDRMLTACPGYRFAAHVWQSTFVLVVRRNAEKPADAEIPTVSAADIYCKMRDISFDGGLMLEYQRLYATFDEFPPP

SEQ ID NO: 9 - protein, 250 aa, human herpesvirus 2
MAGMGKPYGGRPGDAFEGLVQRIRLIVPTTLRGGGGESGPYSPSNPPSRCAFQFHGQDGSDEAFPIEYVLRLMNDWADVPCNPYLRVQNTGVSVLFQGFFNRPHGAPGGAITAEQTNVILHSTETTGLSLGDLDDVKGRLGLDARPMMASMWISCFVRMPRVQLAFRFMGPEDAVRTRRILCRAAEQALARRRRSRRSQDDYGAVAVAAAHHSSGAPGPGVAASGPPAPPGRGPARPWHQAVQLFRAPRP

SEQ ID NO: 10 - protein, 904 aa, human herpesvirus 2
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 11 - protein, 393 aa, human herpesvirus 2
MGRLTSGVGTAALLVVAVGLRVVCAKYALADPSLKMADPNRFRGKNLPVLDRLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 12 - RNA, 1386 nt, Artificial Sequence
  All uridine residues are 1-methyl-pseudouridine - encodes glycoprotein D from Herpes Simplex Virus-2
ggaauaaaagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcaugacccgccugaccgugcuggcccugcuggccggccugcuggccuccucccgcgccaaguacgcccuggccgaccccucccugaagauggccgaccccaaccgcuuccgcggcaagaaccugcccgugcuggaccagcugaccgacccccccggcgugaagcgcguguaccacauccagcccucccuggaggaccccuuccagccccccuccauccccaucaccguguacuacgccgugcuggagcgcgccugccgcuccgugcugcugcacgcccccuccgaggccccccagaucgugcgcggcgccuccgacgaggcccgcaagcacaccuacaaccugaccaucgccugguaccgcaugggcgacaacugcgccauccccaucaccgugauggaguacaccgagugccccuacaacaagucccugggcgugugccccauccgcacccagccccgcugguccuacuacgacuccuucuccgccguguccgaggacaaccugggcuuccugaugcacgcccccgccuucgagaccgccggcaccuaccugcgccuggugaagaucaacgacuggaccgagaucacccaguucauccuggagcaccgcgcccgcgccuccugcaaguacgcccugccccugcgcaucccccccgccgccugccugaccuccaaggccuaccagcagggcgugaccguggacuccaucggcaugcugccccgcuucauccccgagaaccagcgcaccguggcccuguacucccugaagaucgccggcuggcacggccccaagccccccuacaccuccacccugcugccccccgagcuguccgacaccaccaacgccacccagcccgagcuggugcccgaggaccccgaggacuccgcccugcuggaggaccccgccggcaccguguccucccagaucccccccaacuggcacauccccuccauccaggacguggccccccaccacuaacuaguagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 13 - RNA, 1614 nt, Artificial Sequence
  All uridine residues are 1-methyl-pseudouridine - encodes glycoprotein E from Herpes Simplex Virus-2
ggaauaaaagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcaugcgcaugcagcugcugcugcugaucgcccugucccuggcccuggugaccaacucccgcaccuccuggaagcgcgugaccuccggcgaggacguggugcugcugcccgcccccgccggccccgaggagcgcacccgcgcccacaagcugcugugggccgccgagccccuggacgccugcggcccccugcgccccuccuggguggcccuguggcccccccgccgcgugcuggagaccgugguggacgccgccugcaugcgcgcccccgagccccuggccaucgccuacuccccccccuuccccgccggcgacgagggccuguacuccgagcuggccuggcgcgaccgcguggccguggugaacgagucccuggugaucuacggcgcccuggagaccgacuccggccuguacacccuguccguggugggccuguccgacgaggcccgccagguggccuccguggugcuggugguggagcccgcccccgugcccacccccacccccgacgacuacgacgaggaggacgacgccggcguguccgagcgcacccccguguccgugcccccccccacccccccccgccgcccccccguggccccccccacccacccccgcgugauccccgaggugucccacgugcgcggcgugaccgugcacauggagacccccgaggccauccuguucgcccccggcgagaccuucggcaccaacguguccauccacgccaucgcccacgacgacggccccuacgccauggacgugguguggaugcgcuucgacgugcccuccuccugcgccgagaugcgcaucuacgaggccugccuguaccacccccagcugcccgagugccugucccccgccgacgcccccugcgccguguccuccugggccuaccgccuggccgugcgcuccuacgccggcugcucccgcaccaccccccccccccgcugcuucgccgaggcccgcauggagcccgugcccggccuggccuggcuggccuccaccgugaaccuggaguuccagcacgccuccccccagcacgccggccuguaccugugcgugguguacguggacgaccacauccacgccuggggccacaugaccaucuccaccgccgcccaguaccgcaacgccgugguggagcagcaccugccccagcgccagcccgagcccguggagcccacccgcccccacgugcgcgccuaacuaguagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 14 - RNA, 1470 nt, Artificial Sequence
  UL48 of HSV-2 RNA sequence
auggaucugcugguggaugaucuguuugcggaugcggauggcgugagcccgccgccgccgcgcccggcgggcggcccgaaaaacaccccggcggcgccgccgcuguaugcgaccggccgccugagccaggcgcagcugaugccgagcccgccgaugccggugccgccggcggcgcuguuuaaccgccugcuggaugaucugggcuuuagcgcgggcccggcgcugugcaccaugcuggauaccuggaacgaagaucuguuuagcggcuuuccgaccaacgcggauauguaucgcgaaugcaaauuucugagcacccugccgagcgaugugauugauuggggcgaugcgcaugugccggaacgcagcccgauugauauucgcgcgcauggcgauguggcguuuccgacccugccggcgacccgcgaugaacugccgagcuauuaugaagcgauggcgcaguuuuuucgcggcgaacugcgcgcgcgcgaagaaagcuaucgcaccgugcuggcgaacuuuugcagcgcgcuguaucgcuaucugcgcgcgagcgugcgccagcugcaucgccaggcgcauaugcgcggccgcaaccgcgaucugcgcgaaaugcugcgcaccaccauugcggaucgcuauuaucgcgaaaccgcgcgccuggcgcgcgugcuguuucugcaucuguaucuguuucugagccgcgaaauucugugggcggcguaugcggaacagaugaugcgcccggaucuguuugauggccugugcugcgaucuggaaagcuggcgccagcuggcgugccuguuucagccgcugauguuuauuaacggcagccugaccgugcgcggcgugccgguggaagcgcgccgccugcgcgaacugaaccauauucgcgaacaucugaaccugccgcuggugcgcagcgcggcggcggaagaaccgggcgcgccgcugaccaccccgccggugcugcagggcaaccaggcgcgcagcagcggcuauuuuaugcugcugauucgcgcgaaacuggauagcuauagcagcguggcgaccagcgaaggcgaaagcgugaugcgcgaacaugcguauagccgcggccgcacccgcaacaacuauggcagcaccauugaaggccugcuggaucugccggaugaugaugaugcgccggcggaagcgggccugguggcgccgcgcaugagcuuucugagcgcgggccagcgcccgcgccgccugagcaccaccgcgccgauuaccgaugugagccugggcgaugaacugcgccuggauggcgaagaaguggauaugaccccggcggaugcgcuggaugauuuugaucuggaaaugcugggcgauguggaaagcccgagcccgggcaugacccaugauccggugagcuauggcgcgcuggauguggaugauuuugaauuugaacagauguuuaccgaugcgaugggcauugaugauuuuggcggc

SEQ ID NO: 15 - RNA, 906 nt, Artificial Sequence
  UL49 of HSV-2 RNA sequence
augaccucucgccgcuccgucaagucguguccgcgggaagcgccgcgcgggacccacgaggagcuguacuauggcccggucuccccggcggauccagagaguccgcgcgacgacuuccgccgcggcgcuggcccgaugcgcgcgcgcccaaggggcgagguucgcuuucuccauuaugacgaggcuggguaugcccucuaccgggacucgucgucguccgaagacaacgacgagucccgggauaccgcgcgaccgcgucguucggcguccgucgcgggcucucacggccccggccccgcgcgcgcuccuccaccccccgggggccccgugggcgccggcgggcgcucgcacgccccucccgcgcggacccccaaaaugacgcgcggggcgccuaaggcccccgcgaccccggcgaccgacccugcccgcggcaggcgacccgcccaggccgacuccgccgugcuccuagacgcccccgcucccacggccucgggaagaaccaagacacccgcccagggacuggccaagaagcugcacuucagcaccgccccaccgagccccacggcgccguggaccccccggguggccggguucaacaagcgcgucuucugcgccgcggucgggcgccuggcggccacgcacgcccggcuggcggcgguacagcugugggacaugucgcggccgcacaccgacgaagaccucaacgagcuccucgaccucaccaccauucgcgugacggucugcgagggcaagaaccuccugcagcgcgcgaacgaguuggugaaucccgacgcggcgcaggacgucgacgcgaccgcggccgcccggggccgccccgcggggcgugccgccgcgaccgcacgggcccccgcccgcucggcuucccguccccgccgcccccucgag

SEQ ID NO: 16 - RNA, 291 nt, Artificial Sequence
  UL11 of HSV-2 RNA sequence
augggccucgcguucuccggggcgcggcccugcugcugccgacacaacgucaucaucaccgacggcggggaggucgucucgcugaccgcccacgaauuugacgucguggacaucgaguccgaagaagaggguaacuucuacgugccccccgacaugcgcguggucacccgggcgccggggccccaguaccggcgcgcaucggaccccccaucgcgucacaccagacggcgggaccccgacguggcccgcccuccugccacgcucacgcccccacucucugacagcgaauaa

SEQ ID NO: 17 - RNA, 1119 nt, Artificial Sequence
  UL16 of HSV-2 RNA sequen
auggcacagcgggcacucuggcguccccaggcgacgccaggccccccgggagccgcggccccgccgggucaccggggcgccccccccgacgcgcgcgcccccgacccggggcccgaagccgaccucgucgcgcgcaucgccaacucugucuucguguggcgcgucguucggggggacgagcggcuuaagauuuuucggugccucacgguccucaccgagccgcugugccagguggcccuuccggaccccgaccccgagcgugcccuuuucugcgagaucuuccuguaccugacgcggcccaaggcgcugcgccugccuucgaacacguucuuugccauuuucuucuuuaaccgcgagcgccgcuacugcgccaccguccaccuccgcagcguaacgcacccccggaccccgcugcugugcacccuagccuucggccaccuggaagcugccucccccccggaggaaacccccgaccccgccgccgaacagcucgcggacgagcccguggcccacgagcucgacggcgccuaccugguacccacggaaccgcccccgaauccgggggcgugcugugcccuggguccgggggccugguggcaccuccccggcggccgcauauacugcugggccauggaugaugaccuggggucgcucugucccccgggcagccgggcccgccauuuaggauggcugcuauccaggauuaccgaccccccgggaggaggcggcgccugcgccccgacggcccacaucgauuccgccaacgcgcuguggcgcgcccccgccguggcggaggccugucccugcgucgccccgugcaugugguccaauauggcccagcgcacccuggccguccggggggacgcgagccugugucagcuccucuucgggcaccccguggacgcgguuauccugcggcaggcgacccgccgcccgcgcaucacggcccaccugcacgaggucgucgugggccgggacggcgcggaaagcgucauccgcccgaccagcgccggguggcgccucugcguucugucaucguauaccagccgccuguuugcaacgagcugccccgcggucgcgcgggccguggccagagccucaucguccgauuacaaauaa

SEQ ID NO: 18 - RNA, 1599 nt, Artificial Sequence
  UL21 of HSV-2 RNA sequence
auggagcucagcuaugccaccacccugcaccaccgggacguuguguuuuacgucacggcagacagaaaccgcgccuacuuugugugcgggggguccguuuauuccguagggcggccucgggauucucagccgggggaaauugccaaguuuggccuggugguccgggggacaggccccaaagaccgcauggucgccaacuacguacgaagcgagcuccgccagcgcggccugcgggacgugcggcccgugggggaggacgagguguuccuggacagcgugugucugcuaaacccgaacgugagcuccgagcgagacgugauuaauaccaacgacguugaagugcuggacgaaugccuggccgaauacugcaccucgcugcgaaccagcccgggggugcuggugaccggggugcgcgugcgcgcgcgagacagggucaucgagcuauuugagcacccggcgaucgucaacauuuccucgcgcuucgcguacacccccucccccuacguauucgcccuggcccaggcgcaccucccccggcucccgagcucgcuggagccccuggugagcggccuguuugacggcauucccgccccgcgccagccccuggacgcccgcgaccggcgcacggaugucgugaucacgggcacccgcgcccccagaccgauggccgggaccggggccgggggcgcgggggccaagcgggccaccgucagcgaguucgugcaagugaagcacaucgaccguguuguguccccgagcgucucuuccgcccccccgccgagcgcccccgacgcgagucugccgcccccggggcuccaggaggccgccccgccgggccccccgcucagggagcugugguggguguucuacgccggcgaccgggcgcuggaggagccccacgccgagucgggauugacgcgcgaggagguccgcgccgugcauggguuccgggagcaggcguggaagcuguuugggucggugggggcuccgcgggcguuucucggggccgcgcuggcccugagcccgacccaaaagcucgccgucuacuacuaucucauccaccgggagcggcgcaugucccccuuccccgcgcucgugcggcucgucggucgguacauccagcgccacggccuguacguucccgcgcccgacgaaccgacguuggccgaugccaugaacgggcuguuccgcgacgcgcuggcggccgggaccguggccgagcagcuccucauguucgaccuccucccgcccaaggacgugccgguggggagcgacgcgcgggccgacagcgccgcccugcugcgcuuuguggacucgcaacgccugaccccggggggguccgucucgcccgagcacgucauguaccucggcgcguuccugggcguguuguacgccggccacggacgccuggccgcggccacgcauaccgcgcgccugacgggcgugacgucccugguccugaccgugggggacgucgaccggauguccgcguuugaccgcgggccggcgggggcggcuggccgcacgcgaaccgccggguaccuggacgcgcugcuuaccguuugccuggcucgcgcccagcacggccagucuguguga

SEQ ID NO: 19 - RNA, 918 nt, Artificial Sequence
  UL31 of HSV-2 RNA sequence
auguaugacaucgccccccgucgcuccggcucccggcccgggcccggccgcgacaagacccggcggcggucgcgcuuuuccgccgccgggaaccccggcguggagcgccgggccucccgcaagagccugccgucgcacgcgcgcaggcuggagcucugccugcacgagcgccgucgcuaccgcggcuucuucgccgcccucgcccagacgcccuccgaggagaucgccaucgugcgcucgcucuccgugccccuggugaagacgacccccgugucgcuccccuucagccuggaccagaccguggccgacaacugccugacccucucgggcaugggcuacuaccuggggaucgggggcugcugccccgcgugcagcgcgggcgacgggcggcuggccaccgucagccgcgaggcccucauccuggccuucgugcagcagaucaauacgaucuuugagcaccgcaccuuucuggccucccuggucgugcuggccgaccgccacagcaccccccuccaggaccuccuggccgauacgcucggccagcccgagcucuuuuucgugcacacgauccugcgcggagggggggcgugcgacccccgguuucuuuuuuacccggaccccacguacgggggccacaugcuguacgucaucuuccccggcacgucggcccaccugcacuaccggcucaucgaucggaugcugaccgcgugccccggcuaccgguucgccgcccacguguggcagagcacguuugugcucguggucagacgcaacgcagagaaaccagcggacgcggaaauuccgaccgugucggccgcagacauuuauugcaagaugcgggacaucagcuucgacggggggcucaugcuagaguaucaaaggcucuaugcaacauucgacgaguuuccuccgccguag

SEQ ID NO: 20 - RNA, 753 nt, Artificial Sequence
  UL34 of HSV-2 RNA sequence
auggcggggauggggaagcccuacggcggccgcccgggggacgcguucgagggucucguucagcgcaucaggcucauuguucccaccacgcugcgcggcggggguggggagucgggccccuacucgccauccaacccgcccucgagaugugccuuccaguuccacggccaggauggguccgacgaggccuucccgaucgaguacguccugcggcucaugaacgacugggccgaugugcccugcaaccccuaccugcgcgugcagaacaccggcguuucggugcuguuucagggguuuuuuaaccggccccacggcgccccggggggcgcgaucacggcggagcagaccaacgugauucugcacuccaccgagacgacgggacugucccucggagaccuggacgacgucaaggggcgccucggccuggacgcccggccgaugauggccagcauguggaucagcugcuuugugcgcaugccccgggugcagcucgcguuucgguucaugggccccgaggacgccguucgcacgcggcggauccugugucgcgccgccgagcaggcccucgcccgucgccgccgguccaggcggucccaggaugacuacggggcgguggcgguggcggcggcgcaccacucuuccggagcgcccgggccgggggucgccgccucgggcccgccagcgccgcccggacggggaccggcccguccguggcaucaggccgugcaguuguuccgggccccgcguccguga

SEQ ID NO: 21 - RNA, 321 nt, Artificial Sequence
  Cytoplasmic tail of gE protein of HSV-2 RNA sequence
cgcaggcgcuccuggcgggcgguuaaaagccgggccucggcgacgggccccacuuacauucgcguggcggacagcgagcuguacgcggacuggaguucggacagcgagggggagcgcgacgggucccuguggcaggacccuccggagagacccgacucucccuccacaaauggauccggcuuugagaucuuaucaccaacggcuccgucuguauacccccauagcgaggggcguaaaucucgccgcccgcucaccaccuuugguucgggaagcccgggccgucgucacucccaggccuccuauucguccguccucugguaa

SEQ ID NO: 22 - RNA, 1182 nt, Artificial Sequence
  gD of HSV-2 RNA sequence
auggggcguuugaccuccggcgucgggacggcggcccugcuaguugucgcggugggacuccgcgucgucugcgccaaauacgccuuagcagaccccucgcuuaagauggccgaucccaaucgauuucgcgggaagaaccuuccgguuuuggaccagcugaccgacccccccggggugaagcguguuuaccacauucagccgagccuggaggacccguuccagccccccagcaucccgaucacuguguacuacgcagugcuggaacgugccugccgcagcgugcuccuacaugccccaucggaggccccccagaucgugcgcggggcuucggacgaggcccgaaagcacacguacaaccugaccaucgccugguaucgcaugggagacaauugcgcuauccccaucacgguuauggaauacaccgagugccccuacaacaagucguugggggucugccccauccgaacgcagccccgcuggagcuacuaugacagcuuuagcgccgucagcgaggauaaccugggauuccugaugcacgcccccgccuucgagaccgcggguacguaccugcggcuagugaagauaaacgacuggacggagaucacacaauuuauccuggagcaccgggcccgcgccuccugcaaguacgcucucccccugcgcauccccccggcagcgugccucaccucgaaggccuaccaacagggcgugacggucgacagcaucgggauguuaccccgcuuuauccccgaaaaccagcgcaccgucgcccuauacagcuuaaaaaucgccggguggcacggccccaagcccccguacaccagcacccugcugccgccggagcuguccgacaccaccaacgccacgcaacccgaacucguuccggaagaccccgaggacucggcccucuuagaggaucccgccgggacggugucuucgcagauccccccaaacuggcacaucccgucgauccaggacgucgcgccgcaccacgcccccgccgcccccagcaacccgggccugaucaucggcgcgcuggccggcaguacccuggcggcgcuggucaucggcgguauugcguuuuggguacgccgccgcgcucagauggcccccaagcgccuacgucucccccacauccgggaugacgacgcgccccccucgcaccagccauuguuuuacuag

SEQ ID NO: 23 - RNA, 2192 nt, Artificial Sequence
  gB of HSV-2 RNA sequence
augcgcgguggcggauugaucugcgcccugguggugggugcacugguggcugcugucgcuucagcugcuccugcugcuccugcagcuccucgcgcuagugguggcguugcugccaccguggcagcgaacggagguccagcuagccguccucccccagucccgucuccugcaaccacuaaggcacgcaagcguaaaaccaagaaaccgccuaagaggccugaagcaacucccccaccggacgcaaacgcuacuguggcugcuggucacgcuacucucagggcucauuugagagagaucaagguugaaaacgcugacgcccaguucuacgugugcccucccccaacaggugcaacuguugugcaguucgaacagccucgcagguguccuaccaggccugagggucaaaacuacacugaaggcaucgcugucguuuucaaggagaacauugccccauacaaguucaaagcaaccauguacuacaaagacgugacugucucgcaagugugguucggacaccguuacucccaauucauggguaucuucgaggacagggcucccguuccauucgaggaagugaucgauaagauuaacgccaaaggcgugugcagguccacagcaaaguacgucagaaacaacauggagacaacggcuuuccaccgcgacgaucaugagaccgacauggaauugaagccugcgaaaguggcuacaaggacgucgagaggcuggcacaccacugaucugaaguacaaccccuccaggguugaagccuuccauagauacggaacaacggugaacuguaucguugaggaaguggaugcccgcucaguguaccccuacgacgaguucguccuggcuaccggagauuucguuuacaugaguccauucuacggauacagggaggguucgcacaccgaacauacuuccuacgcagcggacagauucaagcagguggaugguuucuacgcucgcgaccugaccacuaaagcacgugcgacuucuccuacaacgaggaaccugcucaccacucccaaguucacagucgcuugggacuggguuccaaagaggccggccgucugcaccaugacuaaauggcaggagguugacgaaaugcugagagcugaauacggcggaagcuuccgcuucuccagcgaugccaucucuacaacguucaccacuaaccugacucaauacucccucagccguguggaccugggugacuguaucggacgcgaugcccgugaggcaauugaccgcauguucgcgcguaaguacaacgcuacacacaucaaagugggccagccacaauacuaccucgcgacggguggcuucuugauugcuuaccagccuuugcugucaaacacucucgcugaauuguacgucagggaguacaugagagaacaagacaggaagccaagaaacgcgacaccggcuccucugagagaggcuccgucugcuaacgccucaguggaacgcaucaagacaaccuccuccauugaguucgcucgucuccaguucaccuacaaccacauccaacgucaugugaacgacaugcugggaaggauugcagucgcguggugcgagcuccagaaccacgaacugacccucuggaacgaggccaggaagcucaacccaaacgcuaucgccucagcaacugucggcaggagaguuagugcgagaauguugggagacgucauggcuguuagcaccugcguuccuguggcccccgauaacgucauuguucaaaacucuaugcgcgucaguucgcguccaggaacuuguuacucccgcccgcucguuagcuuccguuacgaggaccaggguccacugaucgaaggucaacucggcgagaacaacgaauugcgccugacacgugaugcucuggagccuuguacggucggucaccgccguuacuucauuuucggagguggcuacguguacuucgaggaauacgcauacagucaccaguugucgcgcgcugacgugaccacugucuccacauucaucgacuugaacauuacgaugcuggaggaucacgaauucgugccccugggugucuacacacgucaugaaaucaaggacagcggccucuuggauuacaccgagguccagaggagaaaccaacugcacgaccucagauucgcugacaucgauacugugauucgcgcggaugcuaacgcugccauguucgcugg

SEQ ID NO: 24 - RNA, 1251 nt, Artificial Sequence
  gE of HSV-2 RNA sequence
auggccaggggcgccggccugguguucuucgugggcguguggguggugagcugccuggccgccgcccccaggaccagcuggaagagggugaccagcggcgaggacguggugcugcugcccgcccccgccggccccgaggagaggaccagggcccacaagcugcugugggccgccgagccccuggacgccugcggcccccugaggcccagcuggguggcccuguggccccccaggagggugcuggagaccgugguggacgccgccugcaugagggcccccgagccccuggccaucgccuacagcccccccuuccccgccggcgacgagggccuguacagcgagcuggccuggagggacaggguggccguggugaacgagagccuggugaucuacggcgcccuggagaccgacagcggccuguacacccugagcguggugggccugagcgacgaggccaggcagguggccagcguggugcuggugguggagcccgcccccgugcccacccccacccccgacgacuacgacgaggaggacgacgccggcgugagcgagaggacccccgugagcgugcccccccccaccccccccaggaggccccccguggccccccccacccaccccagggugauccccgaggugagccacgugaggggcgugaccgugcacauggagacccccgaggccauccuguucgcccccggcgagaccuucggcaccaacgugagcauccacgccaucgcccacgacgacggccccuacgccauggacgugguguggaugagguucgacgugcccagcagcugcgccgagaugaggaucuacgaggccugccuguaccacccccagcugcccgagugccugagccccgccgacgcccccugcgccgugagcagcugggccuacaggcuggccgugaggagcuacgccggcugcagcaggaccacccccccccccaggugcuucgccgaggccaggauggagcccgugcccggccuggccuggcuggccagcaccgugaaccuggaguuccagcacgccagcccccagcacgccggccuguaccugugcgugguguacguggacgaccacauccacgccuggggccacaugaccaucagcaccgccgcccaguacaggaacgccgugguggagcagcaccugccccagaggcagcccgagcccguggagcccaccaggccccacgugagggcccccccccccgcccccagcgccaggggcccccugagg

SEQ ID NO: 25 - RNA, 1692 nt, Artificial Sequence
  Codon-optimized UL48 of HSV-2 RNA sequence including UTRs and polyA, all uridine residues are 1-methyl-pseudouridine
uaauacgacucacuauaaggacucacuauuuguuuucgcgcccaguugcaaaaagugucgucaccauggaccugcucguugacgauuuguucgcugacgcagauggugugagcccucccccaccgaggccagcugguggcccuaagaacaccccugcugccccuccccuguacgcaacuggcagauugucacaggcgcaacugaugccuaguccaccgaugccugugccucccgcagcucucuucaacagguugcuggacgauuugggcuucagcgcaggacccgcguugugcacaaugcuggauacguggaacgaggaccuguucucuggcuuccccacaaacgcagacauguaccgcgaguguaaauuccugucuacgcucccaucagacgucaucgauuggggcgacgcccacguucccgaaaggucuccaaucgacauuagagcacauggagaugucgcguucccaacccugccggcuacucgcgacgagcucccaucauacuacgaagcgauggcucaguucuucaggggagaguugcgcgcccgugaagaaaguuacagaacagugcuggcuaacuucugcucggcccucuaccgcuacuugcgugcauccgucaggcagcugcacagacaagcgcauaugagggguagaaaccgcgaccugcgcgaaaugcuccguaccacuaucgcugauagguacuacagagagacugcucgccuggcccgugucuuguuccugcaccucuacuuguuccugucacgcgaaauucucugggcugccuacgccgagcaaaugaugcguccagauuuguucgacggccugugcugugaccucgagucguggcgccaguuggcuugucuguuccaaccauugauguucaucaacggaucccugaccguucguggugugccuguggaggcacgccgucuccgcgaauugaaccacauucgugagcaucucaacuugccucugguucguucugcagcugcugaggaaccuggagcuccccucacaacgccaccgguguugcagggcaaccaagcccgcuccagcggauacuucaugcucuugauccgugcuaagcuggacuccuacucuucaguggccaccuccgagggugaaucggucaugcgcgaacacgcuuacucacguggcaggacaagaaacaacuacgguaguacgauugaaggccugcucgaccugcccgacgaugacgaugcaccagcagaggcuggacugguggcuccucgcauguccuuccucagcgccggucagagaccgaggagacucagcaccacugcccccaucaccgaugucucuuugggugacgagcuccguuuggauggcgaggaaguugacaugacuccugcugaugcccucgacgauuucgaccuggaaaugcucggagaugucgagaguccuucgcccgguaugacacacgaccccguuuccuacggugcucuggauguggacgauuucgaguucgaacaaauguucacggacgcaaugggcaucgacgauuucggagguggcgcgggauccggagguggcggaagcgguggcggagguucucaccaccaucaucaccaucaccauuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - RNA, 607 nt, Artificial Sequence
  Codon-optimized UL11 of HSV-2 RNA sequence including UTRs and polyA, all uridine residues are 1-methyl-pseudouridine
uaauacgacucacuauaaggacucacuauuuguuuucgcgcccaguugcaaaaagugucgucaccauggguuuggcuuuuaguggagcuaggccuuguuguuguaggcauaaugugauuauuacugaugguggcgaaguggugucacucacugcucaugaguucgauguuguugauauagaaagugaggaagaagggaauuucuaugucccgccagauaugcggguagugacuagagcccccggcccacaauauagaagagccagcgauccacccucucgacauacucgacgacgagauccagauguagcacggcccccagcgaccuugaccccaccccuguccgauuccgagugagagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcguccaaucaaccucuggauuacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - RNA, 1435 nt, Artificial Sequence
  Codon-optimized UL16 of HSV-2 RNA sequence including UTRs and polyA, all uridine residues are 1-methyl-pseudouridine
uaauacgacucacuauaaggacucacuauuuguuuucgcgcccaguugcaaaaagugucgucaccauggcucaaagagcccuguggagaccacaagccaccccuggaccacccggcgcugccgcuccaccuggacauagaggugcucccccagaugcuagagcuccugauccagguccggaagcagauuugguugccagaauugcgaauagcguguuugucuggagaguugugcgcggugaugaaaggcugaaaaucuuccgcugucugacuguacuuacggaacccuuaugucaagucgcauuacccgauccugauccugaaagagcucuguuuugugaaaucuuucuguaucucacccgcccgaaagcucuccggcucccgucuaauaccuucuucgcgaucuucuucuucaauagagaaaggcgguauugugcaacagugcaucuucggucuguuacccauccgagaacaccacuccucuguacacuggcuuuuggacaucucgaggcggcuucuccacccgaagagacuccugauccagcagcugagcaacuggcagaugaaccaguugcacaugaacuugauggggcauaucucguuccaacugagccaccaccaaacccaggugccuguugcgcguugggaccaggagcuugguggcaucugccaggugggcggaucuauuguugggcuauggacgacgaucucgguagucugugcccacccgggagucgugcuagacaccucggcuggcuucuuucacgcaucacggauccacccggggggggcggggcaugugcucccacagcucauauugacagugcuaaugcccuuuggcgagcuccagcaguugcugaagcuugcccguguguggcuccuuguauguggagcaacauggcgcaacggacacuugcugugcgcggugaugccucacucugccaauugcuguuuggucauccugucgaugcugugauacuucgucaagccacuagacgucccaggauuacagcacaucuccaugaaguggugguaggaagggauggagcagagagugugauuaggcccacguccgcuggcuggagacuguguguguuaagcuccuacacaaguaggcuuuucgcuacuaguuguccggccguugcaagagcugucgcaagggcauccaguucugacuauaaguaggagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcguccaaucaaccucuggauuacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - RNA, 1915 nt, Artificial Sequence
  Codon-optimized UL21 of HSV-2 RNA sequence including UTRs and polyA, all uridine residues are 1-methyl-pseudouridine
uaauacgacucacuauaaggacucacuauuuguuuucgcgcccaguugcaaaaagugucgucaccauggaacuguccuacgcaacaacucuucaucauagagauguagucuucuaugugaccgccgaucggaauagggcauauuucgucuguggcgguagugucuacucugugggcagaccgagggacagccaaccuggcgagaucgcuaaauucgggcucgucgugagggguaccggaccaaaggauagaaugguggcaaauuaugugcgguccgaauugaggcaaagaggucuccgcgauguuagaccuguuggcgaagaugaaguauuucucgauuccguuugcuuguugaauccaaauguuuccucagaacgcgauguaaucaacacuaaugaugucgaggucuuggaugagugucucgcugaguauuguacaagccucaggacgagucccgguguuuuagucacaggaguuagggucagagcacgcgauagagugauugaauuauucgaacaucccgcuauuguaaauaucucuagccgauuugcauauacgccuucuccauauguguuugcucucgcucaagcccaucugccacgucugccauccucacucgaaccgcucguaucuggguuguucgaugggauaccggcaccaagacaaccgcucgaugcucgagaucgccguaccgaugugguuauaaccgggacaagagcaccacggccuauggcgggaacaggcgcaggcggagccggcgcuaaacgcgcuacaguguccgaauuuguacaggucaaacauauagauagaguggucucaccuaguguaagcagugcgccacccccuagugcgccugaugcuucacuucccccgcccggacugcaagaagcggcaccaccaggaccaccccugagagaauuauggugggucuuuuaugcuggagauagggcucuugaagaaccucaugcagaaagcggccugaccagggaagaagugagggcuguccacgguuuuagagaacaagccuggaaauuauucggcucuguuggagcccccagggccuuccugggcgcagcauuagcacucucucccacacagaaacuggcaguguauuauuacuugauacaucgcgaaaggagaaugucuccguuuccggcccuuguacgcuuggugggaagauauauucaaaggcauggacucuaugugccugccccagaugagcccacccucgcugacgcuaugaauggucuguuucgggaugcucuugcugcaggcacaguugcagaacaacuguuaauguuugauuuguuacccccuaaagauguaccaguuggaucugaugcaagagcugauagugcugcacuccucagauucgucgauagccagcggcucacacccgguggcagcgugagcccagaacaugugauguaucucggggcauuucuugguguucuuuaugcugggcauggcagacuugcugccgcgacucacacugcucgguugacagguguaacaagccucguuuugacaguuggcgauguagauaggaugucagccuucgaucgagguccagccggcgcagcaggacgaacuagaacugcaggauaucucgaugcucucuuaacagugugucuugcccgggcucaacaugggcaauccguuuaggagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcguccaaucaaccucuggauuacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 1339 nt, Artificial Sequence
  Codon-optimized gD of HSV-2 RNA sequence including UTRs and polyA, all uridine residues are 1-methyl-pseudouridine
uaauacgacucacuauaaggacucacuauuuguuuucgcgcccaguugcaaaaagugucgucaccaugggccgccucacuagugggguugguacugcugcguuacuggucguggcugucgggcuuagagugguuugugcuaaguaugcacuugcugauccaucacugaaaauggcagacccaaaccgcuucagggguaagaauuugcccgugcuggaucaauugacagauccaccgggcguuaaaagagucuaucauauccaacccagucucgaagauccuuuucaacccccuuccauucccauuaccguauauuaugccguauuagagcgggcuugucguuccguacuuuugcacgcgccauccgaagcaccucaaauugucagaggagcauccgaugaagcuagaaaacauaccuauaaucucacaauagcuugguacagaaugggcgauaacugugccauuccaauuacagucauggaguauacggaauguccuuauaauaaaagucucggaguauguccaauuagaacccaaccacgguggucauauuacgauuccuucagugcgguuucugaagacaaucucggguuucucaugcaugcuccagcuuuugaaacugcaggcacauaucuuagacugguuaaaauuaaugauuggaccgaaauaacccaguucauuuuggaacaucgagcuagggcaagcuguaaauaugcccugccucuccggauuccaccugccgcuuguuugacuagcaaagcuuaucagcaaggaguaaccguagauucuauugguaugcugccuagguucauuccagagaaucaacggacuguugcucucuauucccugaagauugcgggauggcaugggccuaaaccacccuauacaucaacucuuuuaccgcccgaacucucugauacgacaaaugcuacacagccagagcuggugcccgaggauccagaagauagugcucugcuggaagacccggcuggcacagucuccagccaaauuccacccaauuggcauauucccucaauucaagauguggccccucaucaugcuccugcugcaccgaguaauccagguugagagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcguccaaucaaccucuggauuacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - RNA, 1474 nt, Artificial Sequence
  Codon-optimized ICP4 of HSV-2 RNA sequence including UTRs and polyA, all uridine residues are 1-methyl-pseudouridine
uaauacgacucacuauaaggacucacuauuuguuuucgcgcccaguugcaaaaagugucgucaccauggcaguucgcagagcuggccggcaaccaccucgcccucuuggcggugccagaggcggcggcggugccgguccuguaagaggucugggucggccuggagccagaguaggggcuagagcaggccgcagaggagcggcuguaagacccgaccaugcagcggcagugcaugcugggagagguggggauggaguugcaccagaaccagcucgcggggccagaggacgcggugcagguccuggucucuugccugacuuaggucgcggggcccagcaacaauuacugcauuuacgccagcgcggagccggaagggccccaccuggagugagacacgguggugggccucuccgauugggacccggcgccagggguggacgaagaggacaugaacccccuuugcgaccacgugcggaaggguugcccgccgaucaaccugcaccaaggcugagagcuccugcuggggcccgcgagagaggagcugauagaggugcgaauccaaggagaagacggcgccgccaaccacccagaagaaggcggcggccucgggaagcucgacguagaagaaggccuguugcagugggggggggaguugccggucgcagagcucgaggggcacgccgacugagacguaggggcggagccagacgucccggcgcaccugaacgaagggcucgauugagagcuggucggggacggcgaaggcgacgccguagacgccguaggcgacgaugguggcgcagaccugcccgcggaggaggcccuagagggagaggcguuccgggccguuugccgcgggauccagguggggcaggaggagggcuuaggcgcaggccuggcggacgggcaggggcagggcggucaccagcgaggagagcaccugccccuggcgccagaggcagagggcggccagcagcaaggcgaagggccccgccugccagguuagccgcaagggcugcggugagagcccgaagggccggcgccgaugcaccagcccgcggccccgcaagaggccgaaggcaaaggggccgacgaggaaggcgcgcccguagagaaccgggcaggcggggcccugguccgggcgcugcagcagaaccagcaccagcagaacuucgacggagaagaaggagagggccugcgcucccugagccagaacccgccuaggagagcucgcuuucuugcuguccaauuucuauuaaagguuccuuuguucccuaaguccaacuacuaaacugggggauauuaugaagggccuugagcaucuggauucugccuaauaaaaaacauuuauuuucauugcugcguccaaucaaccucuggauuacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - protein, 1320 aa, human herpesvirus 2
MSAEQRKKKTTTTTQGRGAEVTMADEDGGRLRAAAETTGGPGSPDPADGPPPTPNPDRRPAARPGFGWHGGPEENEDEDDDAAADADEAAPASGEAVDEPAADGVVSPRQLALLASMVDEAVRTIPSPPPERDGAEEEAARSPSPPRTPSMRADYGEENDDDDDDDDDDRDAGRWVRGPETTSAVRGAYPDPMASLSPRPPAPRRHHHRHHRRRRAPRRRSAASDSSKSGSSSSASSASSSASSSSSASASSSDDDDDDDAAARAPASAADHAAGGTLGADDEEAGVPARAPGAAPRPSPPRAEPAPGAGAGSARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPAAAAAPLPSAAASPADERAVPAGYGAAGVLAALGRLSAAPASAPAGADDDDDDDGPGGGGGGRRAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSAAAAAADLLFQNQSLRPLLADTVAAADSLAAPASAPREARKRKSPAPARAPPGGAPRPPKKSRADAPRPAAAPPAGAAPPAPPTPPPRPPRPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASAAGRAGTVLAAAGGGVEVVGTAAGLATPPRREPVDMDAELEDDDDGLFGE

(19) United States
(12) Patent Application Publication
(10) Pub. No.: US 2024/0156951 A1
(43) Pub. Date: May 16, 2024

(54) VACCINE COMPOSITIONS AND METHODS FOR TREATING HSV

(71) Applicant: Redbiotec AG, Schlieren (CH)

(72) Inventors: Marina TAMBASCO STUDART, Schlieren (CH); Christian SCHAUB, Schlieren (CH); Corinne JOHN, Schlieren (CH); Martin BÜHLMANN, Schlieren (CH); Martyna WROBLEWSKA, Schlieren (CH); David WILSON, Schlieren (CH)

(21) Appl. No.: 18/280,908

(22) PCT Filed: Mar. 11, 2022
(86) PCT No.: PCT/EP2022/056345

§ 371 Date: Sep. 7, 2023

(30) Foreign Application Priority Data
Mar. 11, 2021 (EP) 21162170.1

(51) Int. Cl. A61K 39/245; A61K 39/00; A61P 37/04

(52) U.S. Cl. CPC A61K 39/245; A61P 37/04; A61K 2039/53; C12N 2710/16622; C12N 2710/16634

(57) ABSTRACT

The present invention relates to a vaccine composition comprising one or more mRNAs encoding a Herpes Simplex Virus (HSV) structural protein or an immunogenic fragment thereof for the treatment or vaccination against HSV.

### CROSS-REFERENCE TO RELATED APPLICATIONS

The present application claims the right of priority of European patent application 21162170 filed with the European Patent Office on 11 Mar. 2021, the entire content of which is incorporated herein for all purposes.

### SEQUENCE LISTING

This application contains a Sequence Listing in computer readable form, which is incorporated herein by reference.

### FIELD OF INVENTION

The present invention relates to a vaccine composition comprising one or more mRNAs encoding Herpes Simplex Virus (HSV) structural proteins or an immunogenic fragment thereof for the treatment of or vaccination against HSV.

### BACKGROUND

Herpes simplex virus is a viral genus of the viral family known as Herpesviridae. The species that infect humans are commonly known as Herpes simplex virus 1 (HSV-1) and Herpes simplex virus 2 (HSV-2), wherein their formal names are Human herpesvirus 1 (HHV-1) and Human herpesvirus 2 (HHV-2), respectively. The initial infection with HSV-1 typically occurs during childhood or adolescence and persists lifelong. Infection rates with HSV-1 are between 40% and 80% worldwide, being higher among people of lower socialeconomic status. In many cases people exposed to HSV-1 demonstrate asymptomatic seroconversion. However, initial infection can also be severe, causing widespread 1 to 2 mm blisters associated with severe discomfort that interferes with eating and drinking to the point of dehydration, last 10 to 14 days, and occur 1 to 26 days after inoculation. Recurrent labial herpes affects roughly one third of the US population, and these patients typically experience 1 to 6 episodes per year. Papules on an erythematous base become vesicles within hours and subsequently progress through ulcerated, crusted, and healing stages within 72 to 96 hours (Cernik et al., 2008, Arch Intern Med., vol. 168, pp. 1137-1144). Global estimates in 2003 assume that 16.2% of the population are infected with HSV-2, being the major cause of genital herpes. The ability of the virus to successfully avoid clearance by the immune system by entering a non-replicating state known as latency leads to lifelong infection. Periodic reactivation from latency is possible and leads to viral shedding from the site of the initial infection. Genital lesions due to herpes are often very painful, and can lead to substantial psychological morbidity. The virus can also be passed from mother to child during birth. Without treatment, 80% of infants with disseminated disease die, and those who do survive are often brain damaged. In addition, genital herpes is associated with an increased risk of HIV acquisition by two- to threefold, HIV transmission on a per-sexual act basis by up to fivefold, and may account for 40-60% of new HIV infections in high HSV-2 prevalence populations (Looker et al., 2008, Bulletin of the World Health Organization, vol. 86, pp. 805-812).

Currently, acyclovir, a synthetic acyclic purine-nucleoside analogue, is the standard therapy for HSV infections and has greatly helped control symptoms. Precursor drugs, valacyclovir (converted to acyclovir) and famciclovir (converted to penciclovir), have been licensed and have better oral bioavailability than acyclovir and penciclovir, respectively. The available drugs have an excellent margin of safety because they are converted by viral thymidine kinase to the active drug only inside virally infected cells. However, HSV can develop resistance to acyclovir through mutations in the viral gene that encodes thymidine kinase by generation of thymidine-kinase-deficient mutants or by selection of mutants with a thymidine kinase unable to phosphorylate acyclovir. Most clinical HSV isolates resistant to acyclovir are deficient in thymidine kinase, although altered DNA polymerase has been detected in some. As HSV can lie latent in neurons for months or years before becoming active, such a therapy may be used to treat symptoms caused by HSV but cannot avoid the periodic reactivation of the virus.

Accordingly, the most effective and economical way to fight HSV would be a vaccine preventing initial infection and/or periodic reactivation of the virus. A lot of effort has been put in the development of such a vaccine in the past several decades. However, so far attempts to develop a potent HSV vaccine have focused on a limited number of antigens that have shown poor performance in clinical trials. Accordingly, there is an urgent need for a vaccine against HSV. Recent attempts have been made to develop an HSV vaccine based on nucleoside modified mRNAs of HSV glycoproteins (US2020/0276300), however these are still in an early stage of development. There remains a need for further HSV vaccinations.

### SUMMARY OF THE INVENTION

The present invention addresses this need and provides novel vaccine compositions comprising one or more mRNAs, wherein each of said mRNAs encodes a Herpes Simplex Virus (HSV) structural protein or an immunogenic fragment thereof selected from the group consisting of UL48; UL48 and UL49; UL11, UL16 and UL21; or UL31 and UL34. Specifically, in the vaccine composition of the invention, the mRNA encodes UL48 having an amino acid sequence which is 80% or more identical to the amino acid sequence of SEQ ID NO: 6, UL49 having an amino acid sequence which is 62% or more identical to the amino acid sequence of SEQ ID NO: 7, UL11 having an amino acid sequence which is 75% or more identical to the amino acid sequence of SEQ ID NO: 1, UL16 having an amino acid sequence which is 72% or more identical to the amino acid sequence of SEQ ID NO: 2, UL21 having an amino acid sequence which is 80% or more identical to the amino acid sequence of SEQ ID NO:3, UL31 having an amino acid sequence which is 85% or more identical to the amino acid sequence of SEQ ID NO: 8, and UL34 having an amino acid sequence which is 70% or more identical to the amino acid sequence of SEQ ID NO: 8.

Preferably, each of the HSV mRNAs in the vaccine composition of the invention is capable of eliciting an immune response when administered in the form of a vaccine composition to a subject.

In addition, the vaccine composition of the invention may further comprise one or more mRNAs encoding a Herpes Simplex Virus (HSV) glycoprotein selected from the group consisting of a) an HSV glycoprotein D (gD) or an immunogenic fragment thereof having an amino acid sequence which is 70% or more identical to the amino acid sequence of SEQ ID NO:11, b) an HSV glycoprotein B (gB) or an immunogenic fragment thereof having an amino acid sequence which is 70% or more identical to the amino acid sequence of SEQ ID NO:10, and c) an HSV glycoprotein E (gE) or an immunogenic fragment thereof having an amino acid sequence which is 70% or more identical to the amino acid sequence of SEQ ID NO: 4 or 80% or more identical to the amino acid sequence of SEQ ID NO: 5, or any combination thereof.

Specific preferred vaccine compositions comprise structural protein UL48 together with glycoproteins gD and/or gB; structural proteins UL 48 and UL49 together with glycoprotein gE, structural proteins UL11, UL16, and UL21 together with glycoproteins gE, gD, and/or gB, and structural proteins UL31 and UL34 together with glycoproteins gD and/or gB.

The vaccine compositions of the invention can optionally comprise mRNAs encoding Herpes Simplex Virus (HSV) glycoproteins that are nucleoside modified mRNAs comprising one or more pseudouridine residues, preferably where the one or more pseudouridine residues comprise m¹ψ(1-methylpseudouridine); m¹acpψ(1-methyl-3-(3-amino-5-carboxypropyl)pseudouridine, ψm (2′-0-methylpseudouridine), m⁵D (5-methyldihydrouridine), m³ψ(3-methylpseudouridine), or any combination thereof.

In these specific embodiments of the vaccine composition, the nucleoside modified mRNAs encoding said immunogenic fragments of glycoproteins are selected from the group consisting of:
HSV gD comprises amino acids 26-331 from HSV-2 strain 333, or a homologous sequence from another HSV strain, preferably wherein the nucleic acid sequence of said nucleoside modified mRNA is as set forth in SEQ ID NO: 12; and
(ii) HSV gE comprises amino acids 24-405 from HSV-2 strain 2.12, or a homologous sequence from another HSV strain, preferably wherein the nucleic acid sequence of said nucleoside modified mRNA is as set forth in SEQ ID NO: 13.

Further, the mRNAs in the vaccine compositions of the invention may encode HSV-1 polypeptides, HSV-2 polypeptides or a mixture thereof.

In addition, each of the mRNAs in the vaccine composition may further comprise a poly-A tail, an m7GpppG cap, 3′-0-methyl-m7GpppG cap, or anti-reverse cap analog, a cap-independent translational enhancer, and/or 5′ and 3′ untranslated regions that enhance translation and/or be codon-optimized (e.g., SEQ ID NOs: 25-30).

Furthermore, in the vaccine compositions of the invention, the mRNAs may be encapsulated in a nanoparticle, lipid, polymer, cholesterol, or cell penetrating peptide, preferably in a liposomal nanoparticle.

The vaccine compositions of the invention may be used in treating or preventing a Herpes Simplex Virus (HSV) infection in a subject. Said HSV infection may be selected from the group consisting of an HSV-1 infection, an HSV-2 infection, a primary HSV infection, a flare, recurrence, or HSV labialis following a primary HSV infection, a reactivation of a latent HSV infection, an HSV encephalitis, an HSV neonatal infection, a genital HSV infection, or an oral HSV infection.

The vaccine composition of the invention may be formulated for intramuscular administration, subcutaneous administration, intradermal administration, intranasal, intravaginal, intrarectal administration, or topical administration, preferably wherein the composition is a vaccine for injection, optionally comprising a pharmaceutically acceptable carrier or adjuvant for injection.

The vaccine composition of the invention may be used as a medicament and/or for therapy.

The vaccine composition of the invention may be used in a method for treating and/or preventing a Herpes Simplex Virus (HSV) infection.

### OVERVIEW OF THE SEQUENCE LISTING

SEQ ID NO: 1 is an exemplary amino acid sequence of UL11 protein of HSV-2.

SEQ ID NO: 2 is an exemplary amino acid sequence of UL16 protein of HSV-2.

SEQ ID NO: 3 is an exemplary amino acid sequence of UL21 protein of HSV-2.

SEQ ID NO: 4 is an exemplary amino acid sequence of gE protein of HSV-2.

SEQ ID NO: 5 is an exemplary amino acid sequence of cytoplasmic tail of gE protein of HSV-2.

SEQ ID NO: 6 is an exemplary amino acid sequence of UL48 protein of HSV-2.

SEQ ID NO: 7 is an exemplary amino acid sequence of UL49 protein of HSV-2.

SEQ ID NO: 8 is an exemplary amino acid sequence of UL31 protein of HSV-2.

SEQ ID NO: 9 is an exemplary amino acid sequence of UL34 protein of HSV-2.

SEQ ID NO: 10 is an exemplary amino acid sequence of gB protein of HSV-2.

SEQ ID NO: 11 is an exemplary amino acid sequence of gD protein of HSV-2.

SEQ ID NO: 12 is an exemplary gD RNA nucleotide sequence fragment of HSV-2 nucleoside modified (all uridine residues are 1-methyl-pseudouridine).

SEQ ID NO: 13 is an exemplary gE RNA nucleotide sequence fragment of HSV-2 nucleoside modified (all uridine residues are 1-methyl-pseudouridine).

SEQ ID NO: 14 is an exemplary UL48 of HSV-2 RNA sequence.

SEQ ID NO: 15 is an exemplary UL49 of HSV-2 RNA sequence.

SEQ ID NO: 16 is an exemplary UL11 of HSV-2 RNA sequence.

SEQ ID NO: 17 is an exemplary UL16 of HSV-2 RNA sequence.

SEQ ID NO: 18 is an exemplary UL21 of HSV-2 RNA sequence.

SEQ ID NO: 19 is an exemplary UL31 of HSV-2 RNA sequence.

SEQ ID NO: 20 is an exemplary UL34 of HSV-2 RNA sequence.

SEQ ID NO: 21 is an exemplary cytoplasmic tail of gE protein of HSV-2 RNA sequence.

SEQ ID NO: 22 is an exemplary gD of HSV-2 RNA sequence.

SEQ ID NO: 23 is an exemplary gB of HSV-2 RNA sequence.

SEQ ID NO: 24 is an exemplary gE of HSV-2 RNA sequence.

SEQ ID NO: 25 is an exemplary codon-optimized UL48 of HSV-2 RNA sequence including exemplary UTRs and exemplary polyA tail, all uridine residues are 1-methyl-pseudouridine.

SEQ ID NO: 26 is an exemplary codon-optimized UL11 of HSV-2 RNA sequence including exemplary UTRs and exemplary polyA tail, all uridine residues are 1-methyl-pseudouridine.

SEQ ID NO: 27 is an exemplary modified (1-methyl-pseudouridine) codon-optimized UL16 of HSV-2 RNA sequence including exemplary UTRs and exemplary polyA tail, all uridine residues are 1-methyl-pseudouridine.

SEQ ID NO: 28 is an exemplary modified (1-methyl-pseudouridine) codon-optimized UL21 of HSV-2 RNA sequence including exemplary UTRs and exemplary polyA tail, all uridine residues are 1-methyl-pseudouridine.

SEQ ID NO: 29 is an exemplary modified (1-methyl-pseudouridine) codon-optimized gD of HSV-2 RNA sequence including exemplary UTRs and exemplary polyA tail, all uridine residues are 1-methyl-pseudouridine.

SEQ ID NO: 30 is an exemplary modified (1-methyl-pseudouridine) codon-optimized ICP4 of HSV-2 RNA sequence including exemplary UTRs and exemplary polyA tail, all uridine residues are 1-methyl-pseudouridine.

SEQ ID NO: 31 is an exemplary amino acid sequence of ICP4 protein of HSV-2 (GenBank Accession Number QI H12398.1).

### BRIEF DESCRIPTION OF THE DRAWINGS

FIG. 1 shows exemplary amino acid sequences with corresponding SEQ ID NOs of the following proteins: UL11 protein of HSV-2, UL16 protein of HSV-2, UL21 protein of HSV-2, gE protein of HSV-2, cytoplasmic tail of gE protein of HSV-2, UL48 protein of HSV-2, UL49 protein of HSV-2, UL31 protein of HSV-2, UL34 protein of HSV-2, gB protein of HSV-2, gD protein of HSV-2.

FIG. 2 shows exemplary nucleotide sequences with corresponding SEQ ID NOs of the following nucleotides: gD RNA nucleotide sequence fragment of HSV-2 nucleoside modified, gE RNA nucleotide sequence fragment of HSV-2 nucleoside modified, UL48 of HSV-2 RNA sequence, UL49 of HSV-2 RNA sequence, UL11 of HSV-2 RNA sequence, UL16 of HSV-2 RNA sequence, UL21 of HSV-2 RNA sequence, UL31 of HSV-2 RNA sequence, UL34 of HSV-2 RNA sequence, Cytoplasmic tail of gE protein of HSV-2 RNA sequence, gD of HSV-2 RNA sequence, gB of HSV-2 RNA sequence, gE of HSV-2 RNA sequence, Codon-optimized UL48 of HSV-2 RNA sequence including UTRs and polyA, Codon-optimized UL11 of HSV-2 RNA sequence including UTRs and polyA, Codon-optimized UL16 of HSV-2 RNA sequence including UTRs and polyA, Codon-optimized UL21 of HSV-2 RNA sequence including UTRs and polyA, Codon-optimized gD of HSV-2 RNA sequence including UTRs and polyA, Codon-optimized ICP4 of HSV-2 RNA sequence including UTRs and polyA.

FIG. 3 shows a Western Blot analysis of an exemplary protein derived from the mRNA UL48 (SEQ ID NO: 25). Western blot layout (L-R): Ladder, Positive control, Negative controls (Collected day 2 and 6), Samples from cells transfected with UL48 Pseudouridine (1-methyl-pseudouridine) mRNA (collected days 1, 2, and 6), Samples from cells transfected with UL48 Uridine mRNA (collected days 1, 2, and 6).

FIG. 4 shows a Western Blot analysis of exemplary proteins derived from the mRNAs UL11, UL16 and UL21 (SEQ ID NOs: 26, 27 and 28). Western blot layout (L-R): Positive control, Negative controls (Collected day 2 and 6), Samples from cells transfected with UL11, UL16, and UL21 Pseudouridine (1-methyl-pseudouridine) mRNA (collected days 1 and 2).

FIG. 5 shows a measurement of IFNγ release using ELISA upon incubation with an exemplary UL48 modified mRNA (SEQ ID NO: 25). OD450 readings from IFNγ ELISA. Graph layout (Top-Bottom): Blank reading from no-sample ELISA, Negative control sample (non-transfected PBMCs), Samples from PBMCs transfected with UL48 Pseudouridine (1-methyl-pseudouridine) mRNA (collected day 3). Error bars indicate Standard Deviation.

FIG. 6 shows a measurement of IFNγ release using ELISA upon incubation with exemplary ICP4 and gD modified mRNAs (SEQ ID NOs: 30 and 29). OD450 readings from IFNγ ELISA. Graph layout (Top-Bottom): Blank reading from no-sample ELISA, Negative control sample (non-transfected PBMCs), Empty Transfection (PBMCs transfected with no mRNA), Samples from PBMCs transfected with ICP4 Pseudouridine mRNA (collected day 3), Samples from PBMCs transfected with gD Pseudouridine (1-methyl-pseudouridine) mRNA (collected day 3). Error bars indicate Standard Deviation.

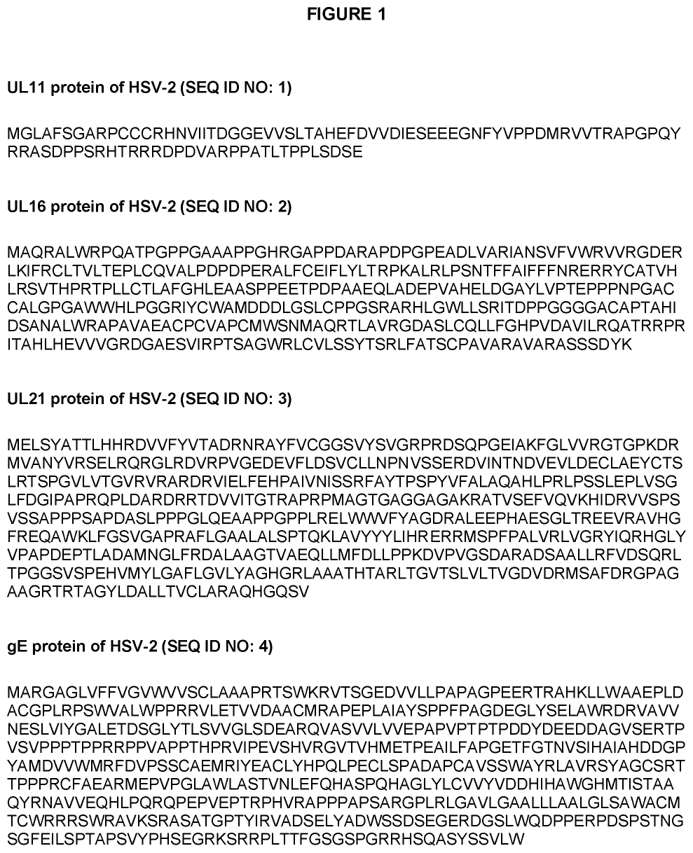

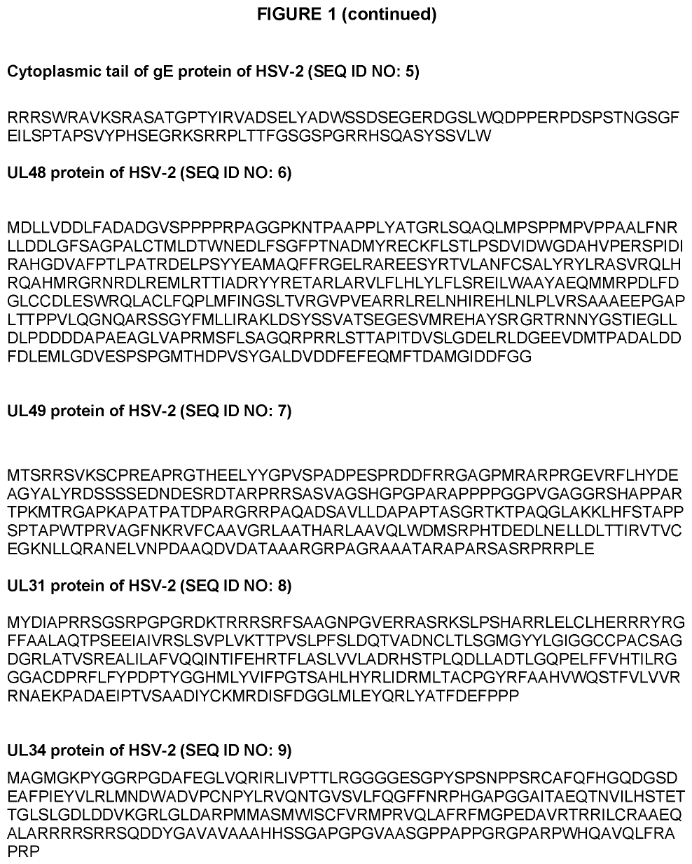

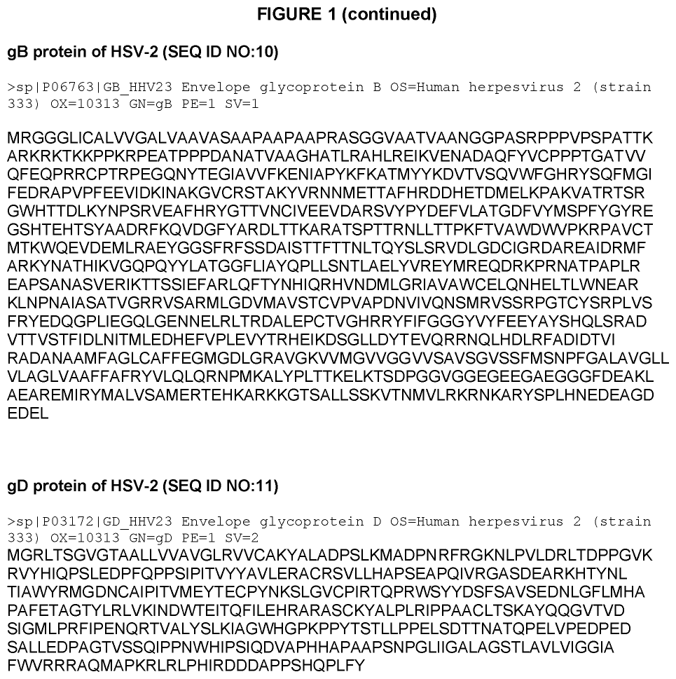

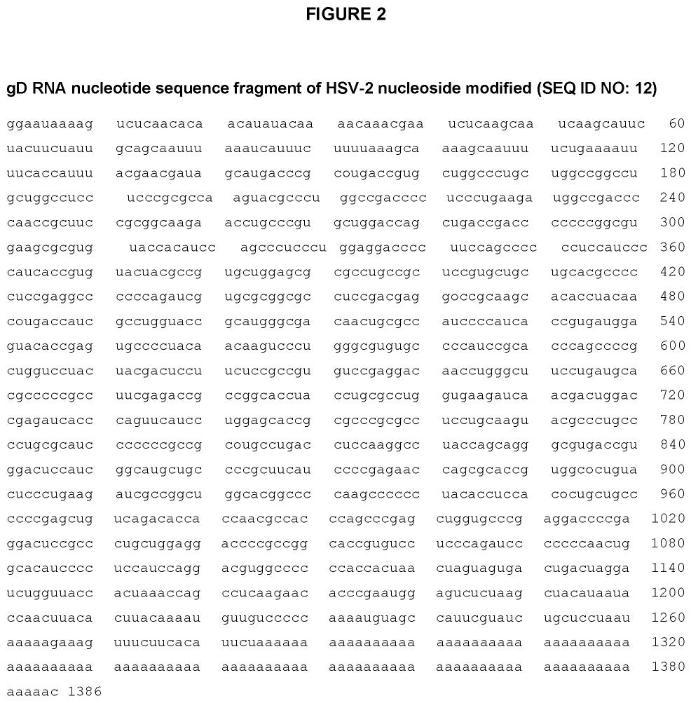

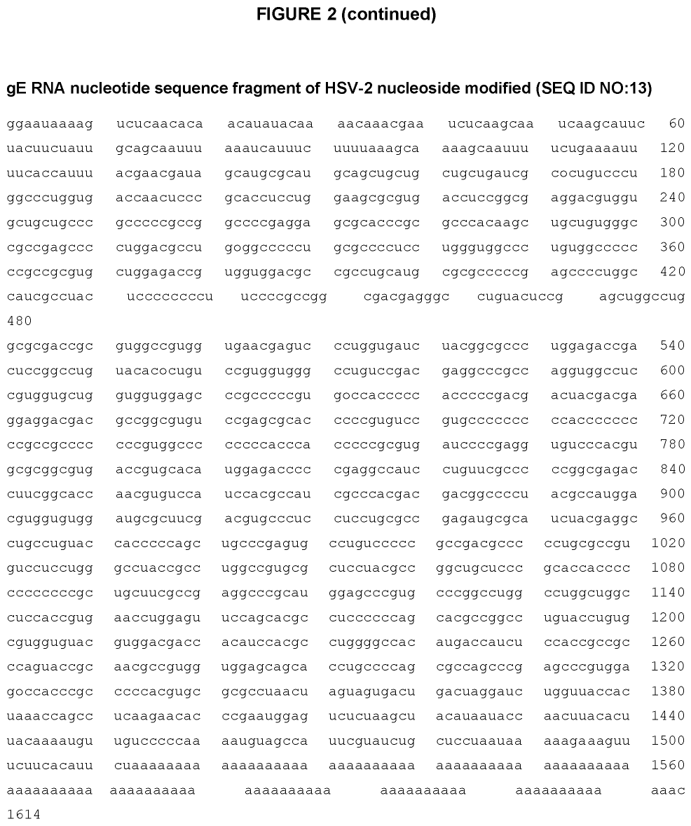

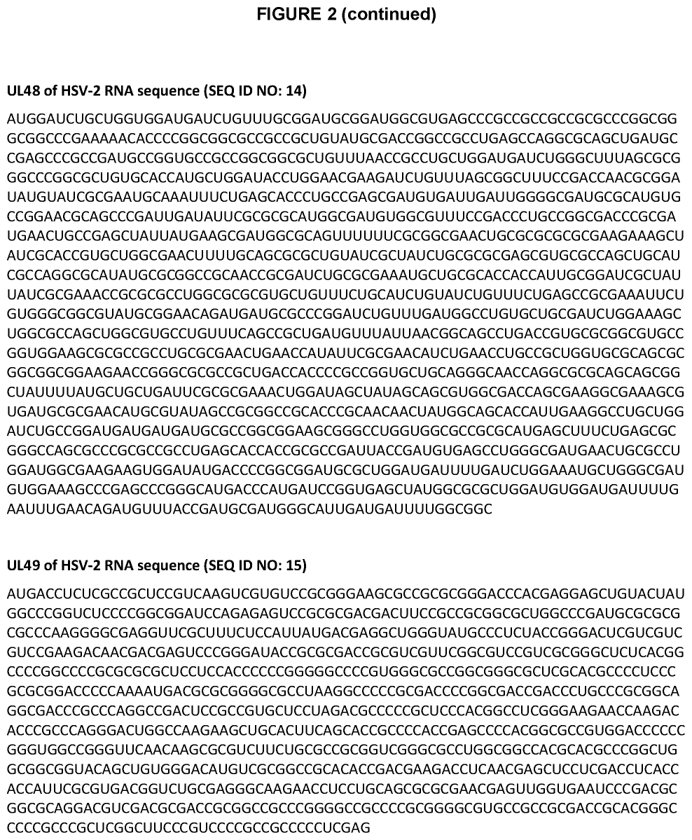

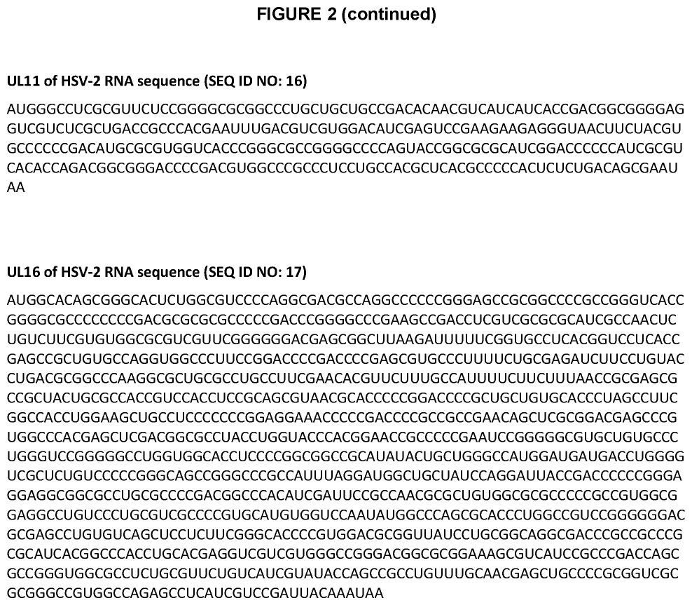

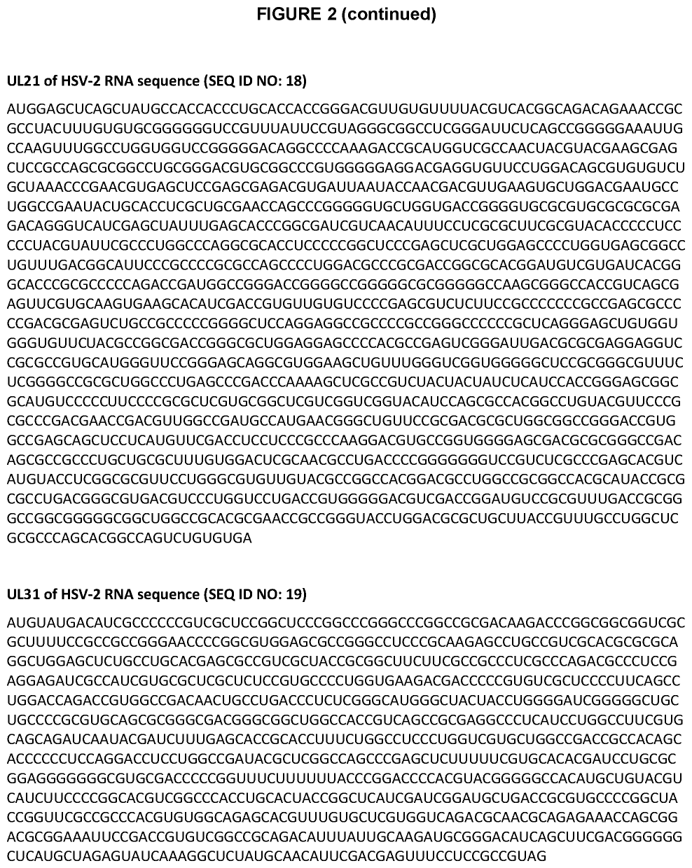

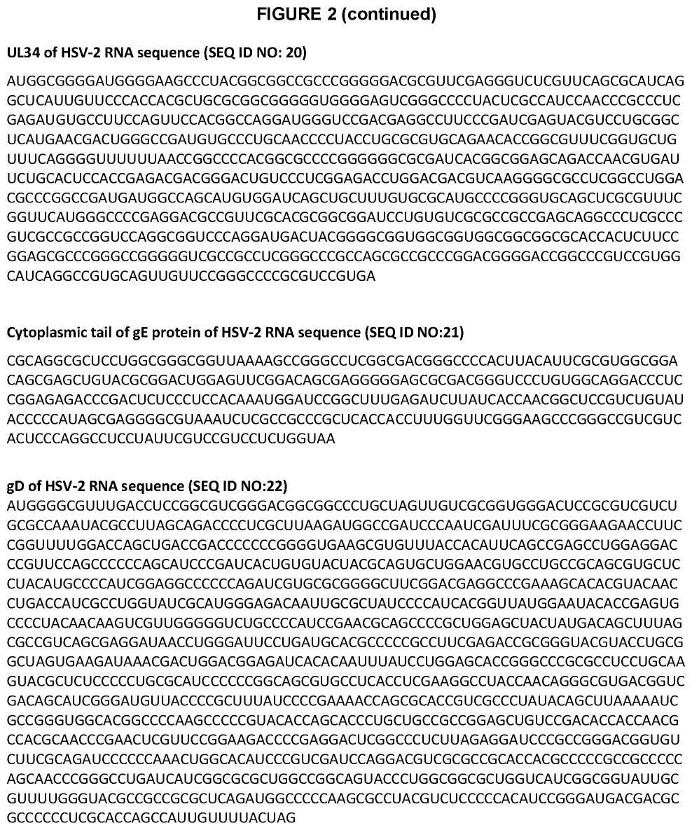

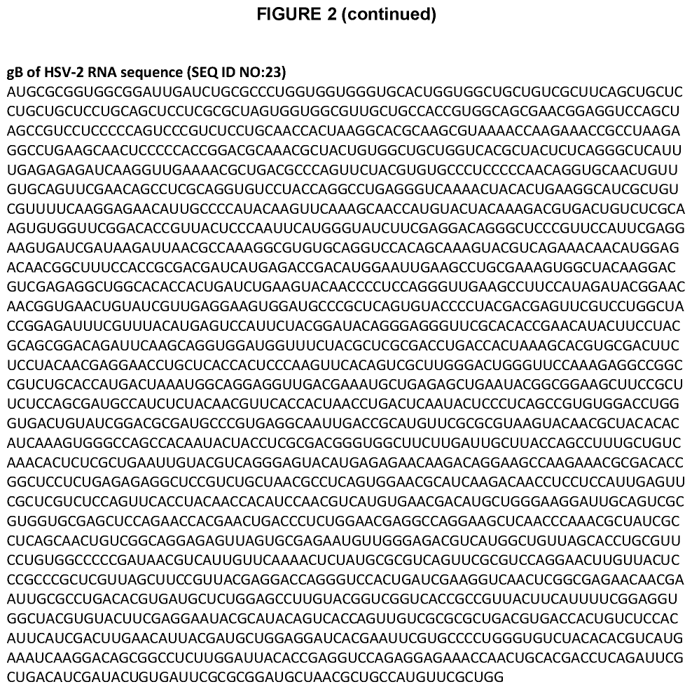

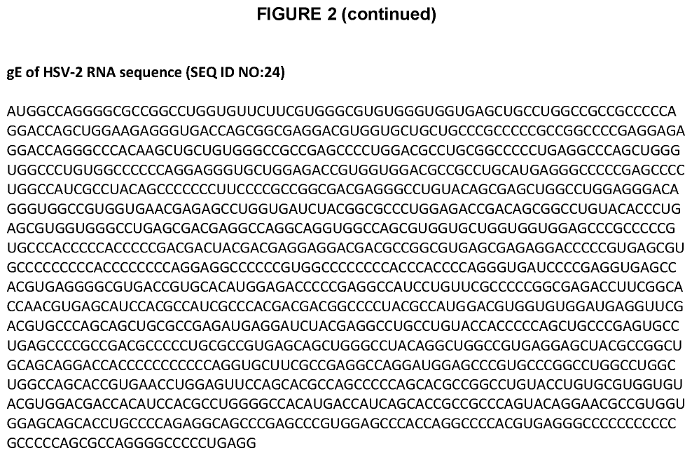

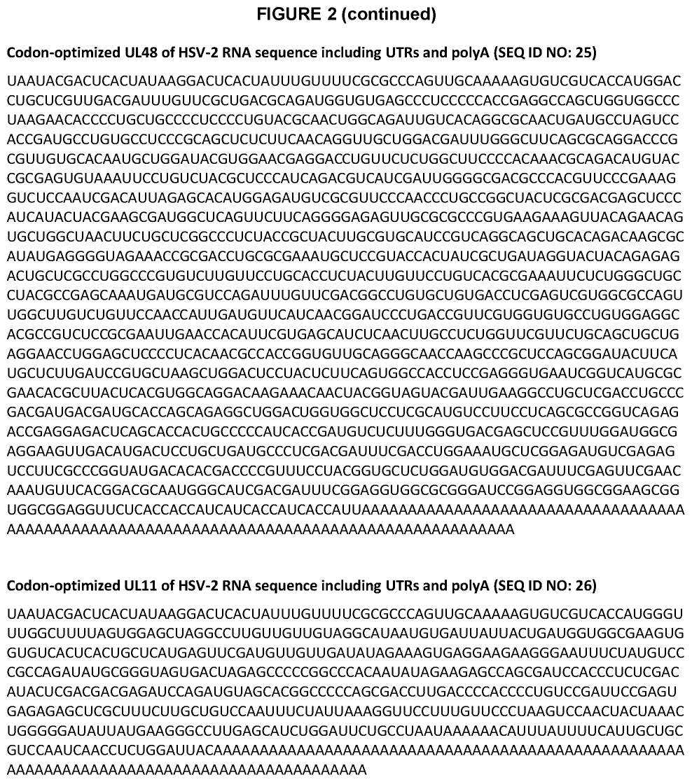

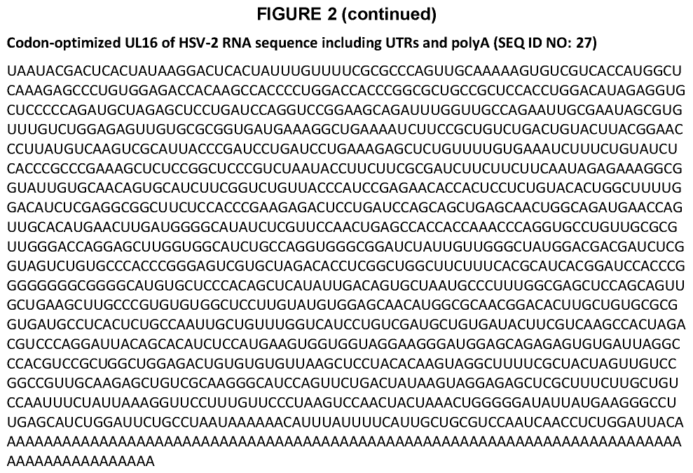

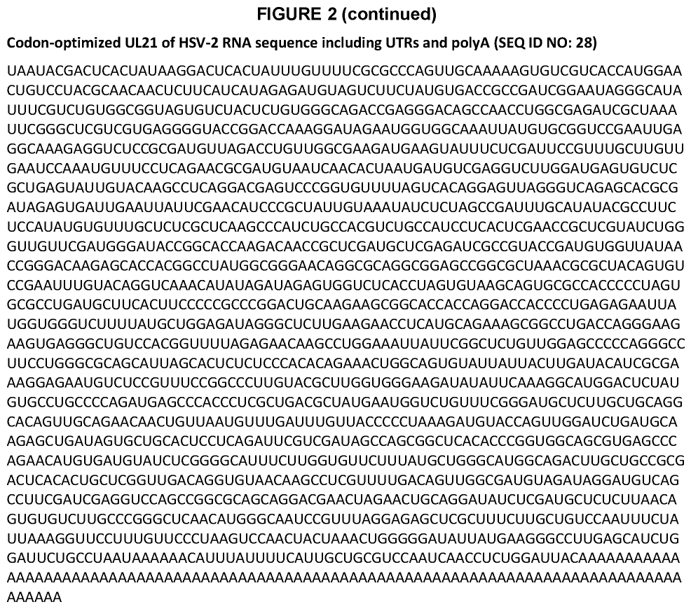

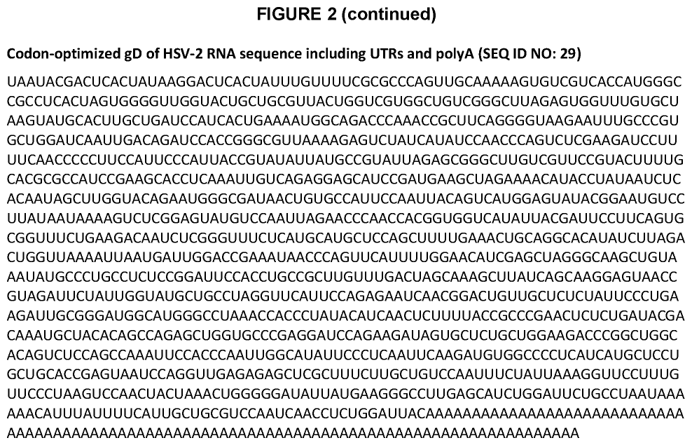

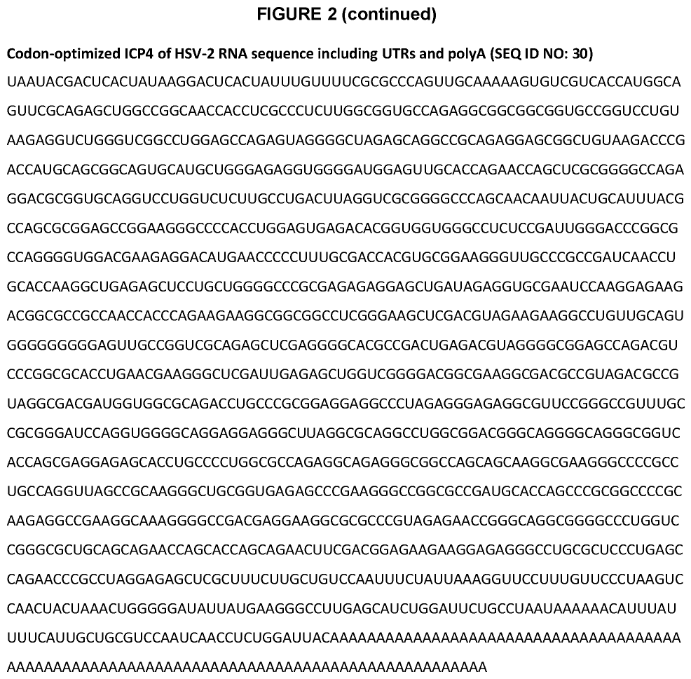

### DETAILED DESCRIPTION

As mentioned above, the present invention provides novel vaccine compositions comprising one or more mRNAs, wherein each of said mRNAs encodes a Herpes Simplex Virus (HSV) structural protein or an immunogenic fragment thereof selected from the group consisting of UL48; UL48 and UL49; UL11, UL16 and UL21; or UL31 and UL34. While research has focused on using glycoproteins such as gE, gC and gD as antigens (see US2020/0276300, e.g., SEQ ID NOs: 4 and 16 therein corresponding to SEQ ID NO: 12 and 13 herein), the inventors surprisingly found that immune reactions to mRNA encoding structural HSV proteins are comparably strong. In addition, as structural proteins are generally not glycosylated, it was not necessary to modify the nucleosides in the mRNAs used.

The term “mRNA” refers to a messenger ribonucleic acid. Generally, such an mRNA encodes a polypeptide and is translated into the protein it encodes in the target cell. To enhance such translation, the mRNA may further comprise a poly-A tail, an m7GpppG cap, 3′-0-methyl-m7GpppG cap, or anti-reverse cap analog, a cap-independent translational enhancer, and/or 5′ and 3′ untranslated regions that enhance translation (e.g., as shown in SEQ ID NOs: 25-30 herein).

A “polypeptide” refers to a molecule comprising a polymer of amino acids linked together by peptide bonds. Said term is not meant herein to refer to a specific length of the molecule and is therefore herein interchangeably used with the term “protein”. When used herein, the term “polypeptide” or “protein” also includes a “polypeptide of interest” or “protein of interest” which is expressed by the expression cassettes or vectors or can be isolated from the host cells of the invention. A polypeptide comprises an amino acid sequence, and, thus, sometimes a polypeptide comprising an amino acid sequence is referred to herein as a “polypeptide comprising a polypeptide sequence”. Thus, herein the term “polypeptide sequence” is interchangeably used with the term “amino acid sequence”.

The term “amino acid” or “aa” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

The term “Herpes Simplex Virus” and “HSV” are used interchangeably herein and refer generally to the viruses of the herpesviral Genus Simplexvirus, i.e. Ateline herpesvirus 1, Bovine herpesvirus 2, Cercopithecine herpesvirus 1, Cercopithecine herpesvirus 2, Cercopithecine herpesvirus 16, Human herpesvirus 1, Human herpesvirus 2, Macropodid herpesvirus 1, Macropodid herpesvirus 2, Saimiriine herpesvirus 1. Preferred viral species of the Genus Simplex virus are viruses infecting humans. Even more preferred viral species are Herpes simplex virus 1 (HSV-1) and Herpes simplex virus 2 (HSV-2) which are also known as human herpesvirus 1 and 2 (HHV-1 and HHV-2), respectively.

The term “vaccine composition” as used herein relates to a composition comprising the mRNAs of the present invention which can be used to prevent or treat a pathological condition associated with HSV in a subject. The “vaccine composition” may or may not include one or more additional components that enhance the immunological activity of the active component or such as buffers, reducing agents, stabilizing agents, chelating agents, bulking agents, osmotic balancing agents (tonicity agents); surfactants, polyols, anti-oxidants; lyoprotectants; anti-foaming agents; preservatives; and colorants, detergents, sodium salts, and/or antimicrobials etc. The vaccine composition may additionally comprise further components typical to pharmaceutical compositions. The vaccine of the present invention is, preferably, for human and/or veterinary use. The vaccine composition may be sterile and/or pyrogen-free. The vaccine composition may be isotonic with respect to humans.

The vaccine composition preferably comprises a therapeutically effective amount of the mRNAs of the invention.

The mRNA of the vaccine composition of the present invention encoding HSV polypeptide UL48 preferably encodes an amino acid sequence which is 80% or more identical to the amino acid sequence of SEQ ID NO: 6, wherein said HSV UL48 mRNA is capable of eliciting an immune response when administered in the form of a vaccine composition to a subject. Preferably, the mRNA is at least 80% identical to SEQ ID NO:14 or a fragment thereof that is at least 200 nucleotides long.

The term “UL48” when used herein relates to the tegument protein VP16 of HSV. SEQ ID NO: 6 depicts exemplarily an amino acid sequence of HSV-2 UL48, also deposited with NCBI GenBank under accession number AHG54712.1. However, the term “UL48” also encompasses UL48 polypeptides having an amino acid sequence which shares a certain degree of identity with the amino acid sequence shown in SEQ ID NO: 6 and also encompasses polypeptides having mutations relative to the reference sequence shown in SEQ ID NO: 6 as described herein. Accordingly, the term “UL48” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 79%, 78%, 77%, 76%, 75%, or preferably 80% or more compared to the amino acid sequence of SEQ ID NO: 6 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 99, 100, 101, 102, 103, 104, 105, 106, 107, 108, 109, 110, 111, 112, 113, 114, 115, 116, 117, 118, 119, 120, 121, 122, 123 or preferably 98 amino acid substitutions, insertions and/or deletions compared to the amino acid sequence of SEQ ID NO: 6. Preferred UL48 proteins translated from mRNA of the invention can form a dimer with UL49 or can form a trimer with UL49 and gE or the cytoplasmic tail of gE.

The mRNA of the vaccine composition of the present invention encoding HSV polypeptide UL49 preferably encodes an amino acid sequence which is 62% or more identical to the amino acid sequence of SEQ ID NO: 7, wherein said mRNA encoding HSV polypeptide UL49 is capable of eliciting an immune response when administered in the form of a vaccine composition to a subject. Preferably, the mRNA is at least 80% identical to SEQ ID NO:15 or a fragment thereof that is at least 200 nucleotides long.

The term “UL49” when used herein relates to the tegument protein VP22 of HSV. SEQ ID NO: 7 depicts exemplarily an amino acid sequence of HSV-2 UL49, also deposited with NCBI GenBank under accession number AKC42813.1. However the term “UL49” also encompasses UL49 polypeptides having an amino acid sequence which shares a certain degree of identity with the amino acid sequence shown in SEQ ID NO: 7 and also encompasses polypeptides having mutations relative to the reference sequence shown in SEQ ID NO: 7 as described herein. Accordingly, the term “UL49” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 74%, 73%, 72%, 71%, 69%, 68%, 67%, 66%, 65%, 64%, 63%, 61%, 60%, 59%, 58%, 57% or preferably 62% or more compared to the amino acid sequence of SEQ ID NO: 2 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 116, 117, 118, 119, 120, 121, 122, 123, 124, 125, 126, 127, 128, 129, 130 or preferably 115 amino acid substitutions, insertions and/or deletions compared to the amino acid sequence of SEQ ID NO: 7. Preferred UL49 proteins translated from the mRNA of the invention can form a complex with UL48 and/or gE or the cytoplasmic tail of gE. Accordingly, preferred UL49 proteins can form a dimer with UL48 or gE or the cytoplasmic tail of gE or can form a trimer with UL48 and gE or the cytoplasmic tail of gE.

In a further preferred embodiment of the present invention mRNA encoding the proteins of the multimeric complex comprising HSV polypeptides UL48, UL49 are comprised in the vaccine composition of the present invention. These may also encode a trimer comprising the cytoplasmic domain of HSV polypeptide gE. In this case the multimeric complex translated from the mRNA of the present invention comprises HSV polypeptides UL48, UL49 and the cytoplasmic domain of gE.

“Sequence identity” or “% identity” refers to the percentage of residue matches between at least two polypeptide sequences aligned using a standardized algorithm. Such an algorithm may insert, in a standardized and reproducible way, gaps in the sequences being compared in order to optimize alignment between two sequences, and therefore achieve a more meaningful comparison of the two sequences. For purposes of the present invention, the sequence identity between two amino acid sequences is determined using the NCBI BLAST program version 2.3.0 (Jan. 13, 2016) (Altschul et al., Nucleic Acids Res. (1997) 25:3389-3402). Sequence identity of two amino acid sequences can be determined with blastp set at the following parameters: Matrix: BLOSUM62, Word Size: 3; Expect value: 10; Gap cost: Existence=11, Extension=1; Compositional adjustments: Conditional compositional score matrix adjustment.

The term “immune response” refers to the ability to induce a humoral and/or cell mediated immune response, preferably but not only in vivo. A humoral immune response comprises a B-cell mediated antibody response. A cell mediated immune comprises a T-cell mediated immune response, including but not limited to CD4+ T-cells and CD8+ T-cells. The ability of an antigen to elicit immune responses is called immunogenicity, which can be humoral and/or cell-mediated immune responses. An immune response of the present invention is preferably an immune response against HSV and even more preferably an immune response against a HSV infection in a subject.

The ability to induce a humoral and/or cell mediated immune response in vivo can be determined using a guinea pig model of genital HSV-2 infection, which accurately mirrors the disease in humans and represents a system to examine pathogenesis and therapeutic efficacy of candidate antiviral compounds and vaccines. It also serves as an ideal system to address the nature of both genital-resident and neural tissue-resident immune memory. Genital infection of guinea pigs results in a self-limiting vulvovaginitis with neurologic manifestations mirroring those found in human disease. Primary disease in female guinea pigs involves virus replication in genital epithelial cells which is generally limited to eight days. During this time, virus reaches sensory nerve endings and is transported by retrograde transport to cell bodies in the sensory ganglia and autonomic neurons in spinal cords. Following a brief period of acute replication at this site, the immune system usually resolves acute virus replication by day 15 post inoculation and the virus is maintained as a lifelong, latent infection of sensory neurons. Following recovering from primary HSV-2 genital infection guinea pigs experience episodic spontaneous recurrent infection and disease. HSV-2 recurrences may manifest as clinically apparent disease with erythematous and/or vesicular lesions on the perineum or as asymptomatic recurrences characterized by shedding of virus from the genital tract. Vaccine efficacy may for example be assessed using the guinea pig genital infection model. Animals may be infected intravaginally with 5×10¹ PFU, 5×10² PFU, 5×10³ PFU, 5×10⁴ PFU, 5×10⁶ PFU, 5×10⁷ PFU, 5×10⁸ PFU, or 5×10 9 PFU and preferably 5×10 5 PFU of HSV-2 (e.g. strain MS). Animals may be immunized prior or post infection one, two, three, four, five or more times. Preferably, at day 15 post infection animals were immunized twice with 15 days interval. In general, any suitable route of administration may be used for immunization. However, animals are preferably immunized intramuscularly. Possible control groups are either mock-immunized with adjuvant-only (e.g. CpG 100 μg/Alum 150 μg) or with PBS (both negative controls), or with the HSV-2 d15-29 mutant virus strain (positive control). Groups that are immunized with vaccine candidates combined with the adjuvant may receive a dose of 0.1 μg, 0.5 μg, 1 μg, 2 μg, 3 μg, 4 μg, 5 μg, 10 μg, 15 μg, 25 μg, 30 μg, 35 μg, 40 μg, 50 μg, 60 μg, 70 μg, 80 μg, 90 μg, 100 μg, 150 μg, 200 μg and preferably 20 μg of the respective mRNA in each immunization round. As a read out vaginal swabs can be collected for evaluation of the frequency and magnitude of recurrent virus shedding, e.g. from day 0 post infection to day 200, day 1 post infection to day 180, day 3 post infection to day 160, day 5 post infection to day 140, day 7 post infection to day 120, day 10 post infection to day 100, day 12 post infection to day 90. Vaginal swabs can be collected every 1, 2, 3, 4, 5, 6, 7, 8, 9, or 10 days. Preferably, vaginal swabs are collected every day, from day 15 post infection to day 85. In the same time interval the severity (scores 0 to 4) and duration of recurrent genital herpetic lesions are scored daily. Preferably, at the end of study the antibody responses as well as the CD4+ and CD8+ T-cell responses are determined.

A variety of routes are applicable for administration of the vaccine composition of the present invention, including, but not limited to, orally, topically, transdermally, subcutaneously, intravenously, intraperitoneally, intramuscularly or intraocularly. However, any other route may readily be chosen by the person skilled in the art if desired.

The exact dose of the vaccine composition of the invention which is administered to a subject may depend on the purpose of the treatment (e.g. treatment of acute disease vs. prophylactic vaccination), route of administration, age, body weight, general health, sex, diet, time of administration, drug interaction and the severity of the condition, and will be ascertainable with routine experimentation by those skilled in the art. The administered dose is preferably an effective dose, i.e. effective to elicit an immune response. In a preferred embodiment, the vaccine composition is administered in two doses of 50-150 μg, preferably 100 μg each 14-42 days apart, preferably 28 days apart.

The vaccine composition of the present invention may be administered to the subject one or more times, such as 2, 3, 4, 5, 6, 7, 8, 9, 10, or more times.

The “subject” as used herein relates to an animal, preferably a mammal, which can be, for instance, a mouse, rat, guinea pig, hamster, rabbit, dog, cat, or primate. Preferably, the subject is a human. However, the term “subject” also comprises cells, preferably mammalian cells, even more preferred human cells. Such a cell may be an immune cell, preferably a lymphocyte.

The mRNA of the vaccine composition of the present invention encoding HSV polypeptide UL11 preferably encodes an amino acid sequence which is 75% or more identical to the amino acid sequence of SEQ ID NO: 1, wherein said HSV UL11 mRNA is capable of eliciting an immune response when administered in the form of a vaccine composition to a subject. Preferably, the mRNA is at least 80% identical to SEQ ID NO:16 or a fragment thereof that is at least 200 nucleotides long.

The term “UL11” when used herein relates to the tegument protein of HSV. SEQ ID NO: 1 depicts exemplarily an amino acid sequence of HSV-2 UL11, also deposited with NCBI GenBank under accession number AHG54674.1. However, the term “UL11” also encompasses UL11 polypeptides having an amino acid sequence which shares a certain degree of identity with the amino acid sequence shown in SEQ ID NO: 1 and also encompasses polypeptides having mutations relative to the reference sequence shown in SEQ ID NO: 1 as described herein. Accordingly, the term “UL11” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 74%, 73%, 72%, 71%, 70% or preferably 75% or more compared to the amino acid sequence of SEQ ID NO: 1 or polypeptides having up to 1, 2, 3, 4, 5, 6, 7, 8, 9, 10, 11, 12, 13, 14, 15, 16, 17, 18, 19, 20, 21, 22, 23, 25, 26, 27, 28, 29 or preferably 24 amino acid substitutions, insertions and/or deletions compared to the amino acid sequence of SEQ ID NO: 1. Preferred UL11 proteins translated from the mRNAs of the invention can form a complex with UL16, UL21 and/or gE or the cytoplasmic tail of gE. Accordingly, preferred UL11 proteins translated from the mRNAs of the invention can form a dimer with UL16 or gE or the cytoplasmic tail of gE, can form a trimer with UL16 and UL21 or with UL16 and gE or the cytoplasmic tail of gE and/or can form a tetramer with UL16, UL21 and gE or the cytoplasmic tail of gE.

The mRNA of the vaccine composition of the present invention encoding HSV polypeptide UL16 preferably encodes an amino acid sequence which is 75% or more identical to the amino acid sequence of SEQ ID NO: 2, wherein said mRNA encoding HSV polypeptide UL16 is capable of eliciting an immune response when administered in the form of a vaccine composition to a subject. Preferably, the mRNA is at least 80% identical to SEQ ID NO:17 or a fragment thereof that is at least 200 nucleotides long.

The term “UL16” when used herein relates to the tegument protein of HSV. SEQ ID NO: 2 depicts exemplarily an amino acid sequence of HSV-2 UL16, also deposited with NCBI GenBank under accession number AHG54679.1. However the term “UL16” also encompasses UL16 polypeptides having an amino acid sequence which shares a certain degree of identity with the amino acid sequence shown in SEQ ID NO: 2 and also encompasses polypeptides having mutations relative to the reference sequence shown in SEQ ID NO: 2 as described herein. Accordingly, the term “UL16” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 71%, 70%, 69%, 68%, 67% or preferably 72% or more compared to the amino acid sequence of SEQ ID NO: 2 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 105, 106, 107, 108, 109, 110, 111, 112, 113, 114, 115, 116, 117, 118, 119, 120, 121, 122, 123, or preferably 104 amino acid substitutions, insertions and/or deletions compared to the amino acid sequence of SEQ ID NO: 2. Preferred UL16 proteins translated from mRNAs of the invention can form a complex with UL11, UL21 and/or gE or the cytoplasmic tail of gE. Accordingly, preferred UL16 proteins translated from mRNAs of the invention can for a dimer with UL21 or UL11, can form a trimer with UL11 and UL21 and/or can form a tetramer with UL11, UL21 and gE or the cytoplasmic tail of gE.

The mRNA of the vaccine composition of the present invention encoding HSV polypeptide UL 21 preferably encodes an amino acid sequence which is 80% or more identical to the amino acid sequence of SEQ ID NO: 3, wherein said mRNA encoding HSV polypeptide UL21 is capable of eliciting an immune response when administered in the form of a vaccine composition to a subject. Preferably, the mRNA is at least 80% identical to SEQ ID NO:18 or a fragment thereof that is at least 200 nucleotides long.

The term “UL21” when used herein relates to the tegument protein of HSV. SEQ ID NO: 3 depicts exemplarily an amino acid sequence of HSV-2 UL21, also deposited with NCBI GenBank under accession number AHG54684.1. However the term “UL21” also encompasses UL21 polypeptides having an amino acid sequence which shares a certain degree of identity with the amino acid sequence shown in SEQ ID NO: 3 and also encompasses polypeptides having mutations relative to the reference sequence shown in SEQ ID NO: 3 as described herein. Accordingly, the term “UL21” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 79%, 78%, 77%, 76%, 75% or preferably 80% or more compared to the amino acid sequence of SEQ ID NO: 3 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, or preferably 134 amino acid substitutions, insertions and/or deletions compared to the amino acid sequence of SEQ ID NO: 3. Preferred UL21 proteins translated from mRNAs of the invention can form a complex with UL11, UL16 and/or gE or the cytoplasmic tail of gE. Accordingly, preferred UL21 proteins can for a dimer with UL16, can form a trimer with UL11 and UL16 and/or can form a tetramer with UL11, UL16 and gE or the cytoplasmic tail of gE.

As mentioned herein, the mRNA encoding the proteins of the multimeric complex comprising HSV polypeptides UL11, UL16, UL21 may further comprise mRNA encoding the HSV glycoprotein gE. In this case the multimeric complex translated from the mRNA of the present invention comprises HSV polypeptides UL11, UL16, UL21, and gE.

The HSV polypeptide UL31 encoded by the mRNA of the vaccine composition of the present invention preferably comprises an amino acid sequence which is 85% or more identical to the amino acid sequence of SEQ ID NO: 8, wherein said mRNA encoding the HSV polypeptide UL31 is capable of eliciting an immune response when administered in the form of a vaccine composition to a subject. Preferably, the mRNA is at least 80% identical to SEQ ID NO:19 or a fragment thereof that is at least 200 nucleotides long.

The term “UL31” when used herein relates to the virion egress protein of HSV. SEQ ID NO: 8 depicts exemplarily an amino acid sequence of HSV-2 UL31, also deposited with NCBI GenBank under accession number AHG54695.1. However, the term “UL31” also encompasses UL31 polypeptides having an amino acid sequence which shares a certain degree of identity with the amino acid sequence shown in SEQ ID NO: 8 and also encompasses polypeptides having mutations relative to the reference sequence shown in SEQ ID NO: 8 as described herein. Accordingly, the term “UL31” encompasses polypeptides having an amino acid sequence identity of 99%, 98%, 97%, 96%, 95%, 94%, 93%, 92%, 91%, 90%, 89%, 88%, 87%, 86%, 84%, 83%, 82%, 81%, 80%, or preferably 85% or more compared to the amino acid sequence of SEQ ID NO: 1 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61 or preferably 46 amino acid substitutions, insertions and/or deletions compared to the amino acid sequence of SEQ ID NO: 8. Preferred UL31 proteins translated from the mRNAs of the invention can form a dimer with UL34.

The HSV polypeptide UL34 encoded by the mRNA of the vaccine composition the present invention preferably comprises an amino acid sequence which is 70% or more identical to the amino acid sequence of SEQ ID NO: 9, wherein said HSV mRNA encoding the polypeptide UL34 is capable of eliciting an immune response when administered in the form of a vaccine composition to a subject. Preferably, the mRNA is at least 80% identical to SEQ ID NO:20 or a fragment thereof that is at least 200 nucleotides long.

The term “UL34” when used herein relates to the virion egress protein of HSV. SEQ ID NO: 9 depicts exemplarily an amino acid sequence of HSV-2 UL34, also deposited with NCBI GenBank under accession number AHG54698.1. However the term “UL34” also encompasses UL34 polypeptides having an amino acid sequence which shares a certain degree of identity with the amino acid sequence shown in SEQ ID NO: 9 and also encompasses polypeptides having mutations relative to the reference sequence shown in SEQ ID NO: 9 as described herein. Accordingly, the term “UL34” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75% 74%, 73%, 72%, 71%, 69%, 68%, 67%, 66%, 65% or preferably 70% or more compared to the amino acid sequence of SEQ ID NO: 2 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 76, 77, 78, 79, 80, 81, 82, 83, 84, 85, 86, 87, 88, or preferably 75 amino acid substitutions, insertions and/or deletions compared to the amino acid sequence of SEQ ID NO: 9. Preferred UL34 proteins translated from the mRNAs of the invention can for a dimer with UL31.

As stated, each mRNA of the invention, may encode a protein containing mutations, such as insertions, deletions and substitutions relative to the reference sequences shown in SEQ ID NO: 1 (UL11), SEQ ID NO: 2 (UL16), SEQ ID NO: 3 (UL21), SEQ ID NO: 4 (gE), SEQ ID NO: 5 (cytoplasmic domain of gE), SEQ ID NO: 6 (UL48), SEQ ID NO: 7 (UL49), SEQ ID NO: 8 (UL31) and SEQ ID NO: 9 (UL34).

In a further preferred embodiment of the present invention, the vaccine composition comprising mRNAs encoding structural HSV polypeptides described above may also encode one or several HSV glycoproteins. Preferred glycoproteins are gE, gB and gD.

The mRNA encoding the HSV glycoprotein gE of the vaccine composition the present invention preferably encodes an amino acid or an immunogenic fragment thereof which is 70% or more identical to the amino acid sequence of SEQ ID NO: 4. Preferably, the mRNA is at least 80% identical to SEQ ID NO:24 or a fragment thereof that is at least 200 nucleotides long.

The term “ICP4” when used herein may refer to the major viral transcription factor 4 of HSV, e.g., deposited with NCBI GenBank under accession number QIH12398.1 (Version 8 Mar. 2020), and having SEQ ID NO: 31 herein. However the term “ICP4” also encompasses ICP4 polypeptides having an amino acid sequence which shares a certain degree of identity with the amino acid sequence shown in SEQ ID NO: 31 and also encompasses polypeptides having mutations relative to the reference sequence shown in SEQ ID NO: 31 as described herein. Accordingly, the term “ICP4” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO: 31 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO: 31. Preferred ICP4 proteins are translated from ICP4 mRNAs (e.g., SEQ ID NO: 30). The mRNA encoding ICP4 may be SEQ ID NO: 30. Preferably, the ICP4 mRNA is at least 80% identical to SEQ ID NO: 30 or a fragment thereof that is at least 200 nucleotides long. In some aspects, the vaccine composition of the invention, comprising at least one mRNA encoding a Herpes Simplex Virus (HSV) glycoprotein selected from the group consisting of a) an HSV glycoprotein D (gD) or an immunogenic fragment thereof having an amino acid sequence which is 70% or more identical to the amino acid sequence of SEQ ID NO:11, b) an HSV glycoprotein B (gB) or an immunogenic fragment thereof having an amino acid sequence which is 70% or more identical to the amino acid sequence of SEQ ID NO:10, and c) an HSV glycoprotein E (gE) or an immunogenic fragment thereof having an amino acid sequence which is 70% or more identical to the amino acid sequence of SEQ ID NO: 4 or 80% or more identical to the amino acid sequence of SEQ ID NO: 5, or any combination thereof; optionally d) an HSV ICP4 or an immunogenic fragment thereof having an amino acid sequence which is 70% or more identical to the amino acid sequence of SEQ ID NO: 31, or any combination thereof. In some further aspects, the vaccine composition of the invention, comprising: (i) UL48 and gD and/or gB, optionally ICP4 (e.g., as above); (ii) UL 48 and UL49 with gE; (iii) UL11, UL16, and UL21 with gE, gD, and/or gB; or (iv) UL31 and UL34 with gD and/or gB.

The term “gE” when used herein may sometimes be referred to as “glycoprotein E”. SEQ ID NO: 4 depicts exemplarily an amino acid sequence of HSV-2 gE, also deposited with NCBI GenBank under accession number AHG54732.1. However the term “gE” also encompasses gE polypeptides having an amino acid sequence which shares a certain degree of identity with the amino acid sequence shown in SEQ ID NO: 4 and also encompasses polypeptides having mutations relative to the reference sequence shown in SEQ ID NO: 4 as described herein. Accordingly, the term “gE” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO: 4 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO: 4. Preferred gE proteins translated from mRNAs of the invention can form a dimer with UL48, a trimer with UL31 and UL34 and a tetramer with UL11, UL16 and UL21.

The mRNA encoding gE may also consist of the cytoplasmic domain of HSV polypeptide gE. Preferably, the gE mRNA is at least 80% identical to SEQ ID NO:21 or a fragment thereof that is at least 200 nucleotides long. Preferably, the mRNA is at least 80% identical to SEQ ID NO:21 or a fragment thereof that is at least 200 nucleotides long

The cytoplasmic domain of gE encoded by the mRNA of the vaccine composition of the present invention preferably comprises an amino acid sequence as set forth in SEQ ID NO: 5. However, it is also envisioned herein that the cytoplasmic domain of gE comprises an amino acid sequence having a sequence identity of 99%, 98%, 97%, 96%, 95%, 94%, 93%, 92%, 91%, 90%, 89%, 88%, 87%, 86%, 85%, 84%, 83%, 82%, 81%, 79%, 78%, 77%, 76%, 75% or preferably 80% or more compared to the amino acid sequence of SEQ ID NO: 5 or polypeptides having up to 1, 2, 3, 4, 5, 6, 7, 8, 9, 10, 11, 12, 13, 14, 15, 16, 17, 18, 19, 20, 21, 22, 24, 25, 26, 27, or preferably 23 amino acid substitutions, insertions and/or deletions compared to the amino acid sequence of SEQ ID NO: 5. Preferred cytoplasmic domains of gE translated from mRNAs of the invention can form a dimer with UL48, a trimer with UL31 and UL34 and a tetramer with UL11, UL16 and UL21.

The mRNA encoding the HSV glycoprotein gD of the vaccine composition the present invention preferably encodes an amino acid or an immunogenic fragment thereof which is 70% or more identical to the amino acid sequence of SEQ ID NO: 11. Preferably, the mRNA is at least 80% identical to SEQ ID NO:22 or a fragment thereof that is at least 200 nucleotides long.

The term “gD” when used herein may sometimes be referred to as “glycoprotein D”. SEQ ID NO: 11 depicts exemplarily an amino acid sequence of HSV-2 gD. However the term “gD” also encompasses gD polypeptides having an amino acid sequence which shares a certain degree of identity with the amino acid sequence shown in SEQ ID NO: 11 and also encompasses polypeptides having mutations relative to the reference sequence shown in SEQ ID NO: 11 as described herein. Accordingly, the term “gD” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO: 11 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO: 11. Preferred gD proteins translated from mRNA of the vaccine composition can form a complex with UL11, UL16 and UL21 proteins translated from mRNA of the vaccine composition. Resulting preferred gD proteins can form a dimer with UL48, a trimer with UL31 and UL34 and a tetramer with UL11, UL16 and UL21.

The mRNA encoding the HSV glycoprotein gB of the vaccine composition the present invention preferably encodes an amino acid or an immunogenic fragment thereof which is 70% or more identical to the amino acid sequence of SEQ ID NO: 10. Preferably, the mRNA is at least 80% identical to SEQ ID NO:23 or a fragment thereof that is at least 200 nucleotides long.

The term “gB” when used herein may sometimes be referred to as “glycoprotein B”. SEQ ID NO: 10 depicts exemplarily an amino acid sequence of HSV-2 gB. However the term “gB” also encompasses gB polypeptides having an amino acid sequence which shares a certain degree of identity with the amino acid sequence shown in SEQ ID NO: 10 and also encompasses polypeptides having mutations relative to the reference sequence shown in SEQ ID NO: 10 as described herein. Accordingly, the term “gB” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO: 10 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO: 10. Preferred gB proteins translated from mRNA of the vaccine composition can form a complex with UL11, UL16 and UL21 proteins translated from mRNA of the vaccine composition. Resulting preferred gB proteins can form a dimer with UL48, a trimer with UL31 and UL34 and a tetramer with UL11, UL16 and UL21.

Also preferred is a nucleoside modified mRNA encoding gE, gB or gD or an immunogenic fragment thereof. Preferred fragments are described in US2020/0276300 and encompass pseudouridine residues, preferably m¹ψ(1-methylpseudouridine); m¹acp³ψ(1-methyl-3-(3-amino-5-carboxypropyl)pseudouridine, ψm (2′-0-methylpseudouridine), m⁵D (5-methyldihydrouridine), m³ψ(3-methylpseudouridine), or any combination thereof. Specifically, the mRNAs of SEQ ID NO: 12 and 13, respectively, are such nucleoside modified gD and gE mRNAs, respectively. Further examples of pseudouridine-modified sequences are shown in SEQ ID NOs: 25-30.

As stated, each mRNA of the invention, may encode a protein containing mutations, such as insertions, deletions and substitutions relative to the reference sequences shown in SEQ ID NO: 1 (UL11), SEQ ID NO: 2 (UL16), SEQ ID NO: 3 (UL21), SEQ ID NO: 6 (UL48), SEQ ID NO: 7 (UL49), SEQ ID NO: 8 (UL31), SEQ ID NO: 9 (UL34), SEQ ID NO: 4 (gE), SEQ ID NO: 5 (cytoplasmic domain of gE), SEQ ID NO:10 (gB) and SEQ ID NO:11 (gD).

In one aspect, the mRNA the of the invention encodes a UL48 protein alone or in combination with an mRNA encoding a glycoprotein selected from the group of gD or gB.

In a further preferred embodiment of the present invention, the mRNAs in the vaccine composition encode two or three structural polypeptides that form a multimeric complex after translation. Additionally, one or more mRNAs encoding glycoprotein gE, gB and/or gD or an immunogenic fragment thereof can be included in the vaccine composition.

Specifically, the vaccine compositions of the invention can comprise mRNA encoding UL48 and UL49, which when translated can form a complex. Alternately, the vaccine compositions of the invention can comprise mRNA encoding UL48, UL49 and glycoprotein gE, all of which can form a complex when translated.

In another embodiment, the vaccine compositions of the invention can comprise mRNA encoding UL11, UL16 and UL21, which when translated can form a complex. The vaccine compositions of the invention comprising mRNA encoding UL11, UL16 and UL21 can further comprise one or more mRNAs encoding glycoprotein gE, gB or gD.

Alternately, the vaccine compositions of the invention can comprise mRNA encoding UL31 and UL34, which when translated can form a complex. The vaccine compositions of the invention comprising mRNA encoding UL31 and UL34 can further comprise one or more mRNAs encoding glycoprotein gB or gD.

The mRNA in the vaccine compositions can encode HSV-1 polypeptides, HSV-2 polypeptides or a mixture thereof.

The vaccine composition of the invention may further comprise a pharmaceutically acceptable carrier or adjuvant.

The terms “carrier” and “excipient” are used interchangeably herein. Pharmaceutically acceptable carriers include, but are not limited to diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), anti-foaming agents (e.g. Simethicone), thickening agents (e.g. methylcellulose or hydroxyethylcellulose), sweetening agents (e.g. sorbitol, saccharin, aspartame, acesulfame), flavouring agents (e.g. peppermint, lemon oils, butterscotch, etc), humectants (e.g. propylene, glycol, glycerol, sorbitol). Further pharmaceutically acceptable carriers are (biodegradable) liposomes; microspheres made of the biodegradable polymer poly(D,L)-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable excipients are inter alia described in Remington's Pharmaceutical Sciences, 15th Ed., Mack Publishing Co., New Jersey (1991) and Bauer et al., Pharmazeutische Technologie, 5th Ed., Govi-Verlag Frankfurt (1997). The person skilled in the art will readily be able to choose suitable pharmaceutically acceptable carriers, depending, e.g., on the formulation and administration route of the pharmaceutical composition.

The term “adjuvant” as used herein refers to a substance that enhances, augments or potentiates the host's immune response (antibody and/or cell-mediated) to an antigen or fragment thereof. Exemplary adjuvants for use in accordance with the present invention include inorganic compounds such as alum, aluminum hydroxide, aluminum phosphate, calcium phosphate hydroxide, the TLR9 agonist CpG oligodeoxynucleotide, the TLR4 agonist monophosphoryl lipid (MPL), the TLR4 agonist glucopyranosyl lipid (GLA), the water in oil emulsions Montanide ISA 51 and 720, mineral oils, such as paraffin oil, virosomes, bacterial products, such as killed bacteria Bordetella pertussis, Mycobacterium bovis, toxoids, nonbacterial organics, such as squalene, thimerosal, detergents (Quil A), cytokines, such as IL-1, IL-2, IL-10 and IL-12, and complex compositions such as Freund's complete adjuvant, and Freund's incomplete adjuvant. Generally, the adjuvant used in accordance with the present invention preferably potentiates the immune response to the multimeric complex of the invention and/or modulates it towards the desired immune responses.

The term “pharmaceutically acceptable” means a non-toxic material that does not interfere with the effectiveness of the biological activity of the multimeric complex according to the present invention.

Use of the Vaccine Composition

The present invention also pertains to the use of the vaccine composition in a method of inducing an immune response against HSV in a subject.

In a preferred embodiment of the present invention the vaccine composition is used for the treatment, prevention or amelioration of HSV infection or preventing reactivation of HSV. HSV infection can be selected from the group consisting of an HSV-1 infection, an HSV-2 infection, a primary HSV infection, a flare, recurrence, or HSV labialis following a primary HSV infection, a reactivation of a latent HSV infection, an HSV encephalitis, an HSV neonatal infection, a genital HSV infection, and an oral HSV infection.

Accordingly, the vaccine composition may be used in fighting diseases caused by HSV and/or related symptoms. It is also envisaged that the vaccine composition of the present invention may be used for clearing the virus in a subject, i.e. after treatment no HSV can be detected in a suitable sample obtained from the subject using suitable methods known to those of ordinary skill in the art, e.g. PCR, ELISA etc. Thus, the vaccine composition of the present invention may be used to block primary infection, stop primary disease, block virus reactivation and re-infection, and to block latency.

To reduce the chance of genital herpes a prophylactic vaccine to prevent the first HSV infection of the mother is desirable, whereas an effective therapy is needed in the case a mother is diagnosed with an active HSV infection. A multimeric complex of the present invention may be applied as a prophylactic vaccine, e.g. for expectant mothers or children, or as a therapeutic vaccine in seropositive women to prevent subclinical reactivation at the time of delivery.

In a further preferred embodiment of the present invention the vaccine composition is used in a method for inducing an immune response against HSV-1 or HSV-2 in a subject.

The terms “polynucleotide”, “nucleotide sequence” or “nucleic acid molecule” are used interchangeably herein and refer to a polymeric form of nucleotides which are usually linked from one deoxyribose or ribose to another. The term “polynucleotide” preferably includes single and double stranded forms of DNA or RNA. A nucleic acid molecule of this invention may include both sense and antisense strands of RNA (containing ribonucleotides), cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The vaccine composition of the invention may be used in a prime boost regimen. In the prime boost regimen, a prime/boost vaccine is used which is composed of two or more types of vaccine including a vaccine used in primary immunization (prime or priming) and a vaccine used in booster immunization (boost or boosting). The vaccine used in primary immunization and the vaccine used in booster immunization may differ from each other. Primary immunization and boosting immunization may be performed sequentially, this is, however, not mandatory. The prime/boost regimen includes, without limitation, e.g. mRNA prime/protein boost. However, the boosting composition can also be used as priming composition and said priming composition is used as boosting composition.

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

The term “about” or “approximately” as used herein means within 20%, preferably within 10%, and more preferably within 5% of a given value or range. It includes also the concrete number, e.g., about 20 includes 20.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol 11976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

### EXAMPLES

The following hypothetical Examples illustrate the invention, but are not to be construed as limiting the scope of the invention.

### Example 1

PBMC from four HSV-2-infected individuals and two uninfected individuals were thawed and left rest overnight. Cells were seeded onto plates at 5×10⁵ cells/well and subsequently stimulated with 5 μg/mL of HSV-2 UL48 mRNA alone or with 5 μg/mL UL49 mRNA for 48h. Supernatants were thereafter collected and analyzed for the secretion of IFN-γ with a Luminex instrument. The background signal (generated from buffer-stimulated cells) was subtracted from each well and results were expressed as μg/ml.

### Example 2

Splenocytes from HSV-2 infected and control guinea pigs (1×10⁵ cells) were mixed with 10 μg/mL of HSV-2 UL31 mRNA and 10 μg/mL UL34 mRNA. Cells were then transferred onto ELISPOT anti-interferon gamma (IFN-γ) antibody-coated plates (Multiscreen HTS Plates; Millipore) and incubated for 20 h. Plates were thereafter developed according to standard ELISPOT protocols and the IFN-γ secreting cells were quantified as spots using an automated reader. Unstimulated cells and 20 μg/mL of PHA were used as negative and positive controls, respectively.

### Example 3

PBMC from fourteen HSV-2-infected and six uninfected individuals were thawed and left rest overnight. Cells were plated onto ELISPOT anti-interferon gamma (IFN-γ) antibody coated plates at 2×10⁵ cells/well. Cells were subsequently stimulated with 5 μg/mL of HSV-2 UL31 mRNA and 5 μg/mL of HSV-2 UL34 mRNA for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×10⁵ PBMC.

### Example 4

PBMC from four HSV-2-infected and two uninfected individuals were thawed and left rest overnight. Cells were plated onto ELISPOT anti-interferon gamma (IFN-γ) antibody coated plates at 2×10⁵ cells/well. Cells were subsequently stimulated with 5 μg/mL of HSV-2 UL48 mRNA alone or with 5 μg/mL UL49 mRNA, for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×10⁵ PBMC.

### Example 5

PBMC from fourteen HSV-2-infected and six uninfected individuals were thawed and left rest overnight. Cells were plated onto ELISPOT anti-interferon gamma (IFN-γ) antibody coated plates at 2×10⁵ cells/well. Cells were subsequently stimulated with 5 μg/mL of HSV-2 UL48 mRNA alone or in combination with 5 μg/mL UL49 mRNA for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×10⁵ PBMC.

### Example 6

PBMC from four HSV-2-infected and two uninfected individuals were thawed and left rest overnight. Cells were plated onto ELISPOT anti-interferon gamma (IFN-γ) antibody coated plates at 2×10⁵ cells/well. Cells were subsequently stimulated with 5 μg/mL of HSV-2 UL11 mRNA, 5 μg/mL UL16 mRNA and 5 μg/mL UL21 mRNA, or the respective mRNA encoding UL11, UL16 or UL21 normalized to the amount of the single proteins in the combination, for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×10⁵ PBMC.

### Example 7

PBMC from fourteen HSV-2-infected and six uninfected individuals were thawed and left rest overnight. Cells were plated onto ELISPOT anti-interferon gamma (IFN-γ) antibody coated plates at 2×10⁵ cells/well. Cells were subsequently stimulated with 5 μg/mL of HSV-2 UL11 mRNA, 5 μg/mL UL16 mRNA and 5 μg/mL UL21 mRNA for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×10⁵ PBMC.

### Example 8

PBMC from four HSV-2-infected individuals and two uninfected individuals were thawed and left rest overnight. Cells were seeded onto plates at 5×10⁵ cells/well and subsequently stimulated with 5 μg/mL of HSV-2 UL31 mRNA and 5 μg/mL of HSV-2 UL34 mRNA for 48 h. Supernatants were thereafter collected and analyzed for the secretion of IFN-γ with a Luminex instrument. The background signal (generated from buffer-stimulated cells) was subtracted from each well and results were expressed as μg/ml.

### Example 9

PBMC from four HSV-2-infected individuals and two uninfected individuals were thawed and left rest overnight. Cells were seeded onto plates at 5×10⁵ cells/well and subsequently stimulated with 5 μg/mL of HSV-2 UL11 mRNA, 5 μg/mL UL16 mRNA and 5 μg/mL UL21 mRNA for 48 h. Supernatants were thereafter collected and analyzed for the secretion of IFN-γ with a Luminex instrument. The background signal (generated from buffer-stimulated cells) was subtracted from each well and results were expressed as μg/ml.

### Example 10: mRNA Transfection and Western Blot

Methods:

HEK 293T cells were seeded at a concentration of 0.4×10⁶/ml in 12 well plates containing RPMI media and 10% FBS, and incubated at 37° C. and 5% CO2. The next day the cells were transfected using Invitrogen Lipofectamine MessengerMAX Transfection kit. 2-4.5 μl of 1 μg/μl mRNA (SEQ ID NO: 25 and SEQ ID NOs: 26, 27 and 28) was added per well. The empty transfection wells had only the transfection reagent added, nothing was added to the negative control wells.

The cells were harvested over the following days. To do this, the media was removed from the wells and 70 μl of chilled Thermo Scientific RIPA Lysis and Extraction Buffer, along with 10 μl 7× complete, EDTA-free Protease Inhibitor Cocktail was added to each well. The plate was incubated at 4° C. for 2-3 minutes, and the cells were then detached using a cell scraper. The cell-buffer mix was transferred to a 1.5 ml Eppendorf tube and incubated on ice. 70 μl of 2× Biorad Laemli buffer containing 50 mM DTT was added to each tube, and the samples were boiled at 90° C. for 5 minutes. For the positive controls, a sample of the recombinant protein(s) was added to RIPA buffer and proteinase and treated in an identical way to the other samples.

Prior to loading the samples on a gel, 1 μl of Thermo Scientific Pierce Universal Nuclease for cell lysis was added to each. 20 μl of each sample was then loaded onto an Invitrogen Bolt 4-12% Bis-Tris Plus gel, which was run at 90V for 40 minutes. Following this, the samples were transferred using an iBlot 2 system and iBlot 2 mini PVDF Transfer Stacks. The settings used were: 20V for 1 minute, 23V at 4 minutes, and 25V for 90 seconds.

The membrane was then blocked overnight at 4° C. in 5% BSA TBS containing 0.1% Tween-20. Primary antibodies, which were either purchased commercially or produced in-house, were added to the blocking buffer at a 1:1000 concentration and incubated at room temperature while being gently shaken for 1 hour. The membrane was then washed 3× with TBS containing 0.1% Tween-20. Following this, the secondary antibody was added in a 1:5000 concentration in blocking buffer and incubated at room temperature while being gently shaken for 1 hour. The membrane was again washed 3× with BST containing 0.1% Tween-20.

To perform the imaging, 300 μl of SuperSignal West Femto Maximum Sensitivity Substrate was applied to the membrane and it was imaged using a Full frame camera with a 100 mm F/2.8 lens and dark box.

Results:

FIG. 3 (Western Blot of the protein derived from the mRNA UL48—SEQ ID NO: 25) clearly shows expression of UL48 protein following transfection of the mRNA construct. Highest expression is apparent 1 day after transfection, and then falls with time. This indicates robust protein expression as a result of the application of our UL48 mRNA constructs (e.g., SEQ ID NO: 25).

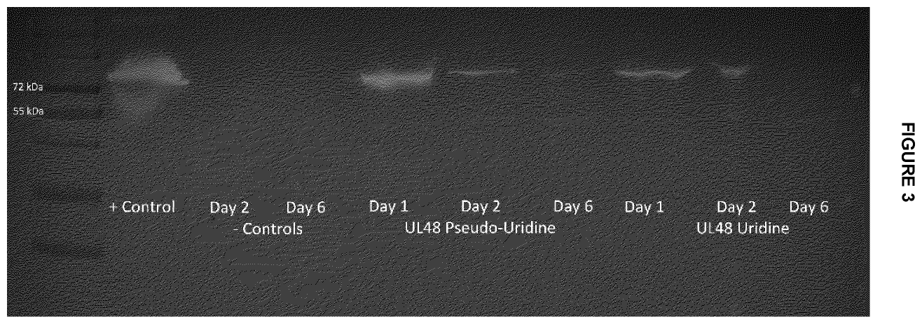

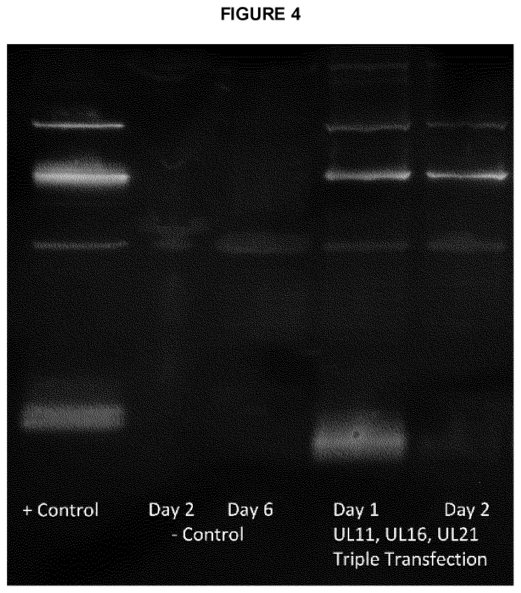

FIG. 4 (Western Blot of the protein derived from the mRNAs UL11, UL16 and UL21— SEQ ID NOs: 26, 27 and 28) clearly shows expression of UL11, UL16, and UL21 following co-transfection of our mRNA constructs. As observed for UL48, expression is highest one day post-transfection.

### Example 11: Immunogenicity Data Using PBMCs

Methods:

PBMC Propagation and IFNγ ELISA Protocol

PBMCs collected from HSV-2+ donors were thawed and grown overnight in RPMI containing 10% FBS in 12 well plates at a concentration of 1×10⁶ cells/ml. The next day the cells were transfected using Invitrogen Lipofectamine MessengerMAX Transfection kit. 1-2 μl of 1 μg/μl mRNA (SEQ ID NO: 25, SEQ ID NO: 2 and SEQ ID NO: 30) was added per well. The empty transfection wells had only the transfection reagent added, nothing was added to the negative control wells.

The samples were harvested 3 days post-transfection. The supernatant was centrifuged at 500RCF for 6 minutes. Afterwards the IFNγ levels were assessed using an Invitrogen Human IFN Gamma Uncoated ELISA kit and F96 Maxisorp Nunc-Immuno plates. OD450 measurements were performed in a Tecan Infinite M Plex plate reader.

Results:

The ELISA results show the secretion of IFNγ in PBMCs from HSV 2+ donors triggered by the pseudouridine UL48, gD and ICP4 mRNAs. These data indicate that specific immune responses are triggered by the expression of the applied HSV-2 mRNAs. No mRNA or transfection reagent was added to the negative control wells. For the blank wells no biological sample was added during the ELISA.

FIG. 5 shows a measurement of IFNγ release using ELISA upon incubation with the UL48 modified mRNA, no mRNA or transfection reagent was added to the negative control wells, for the blank wells no biological sample was added during the ELISA.

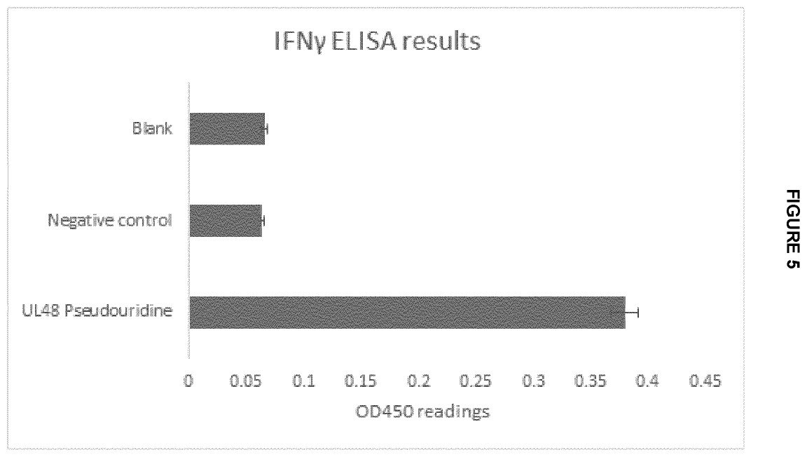

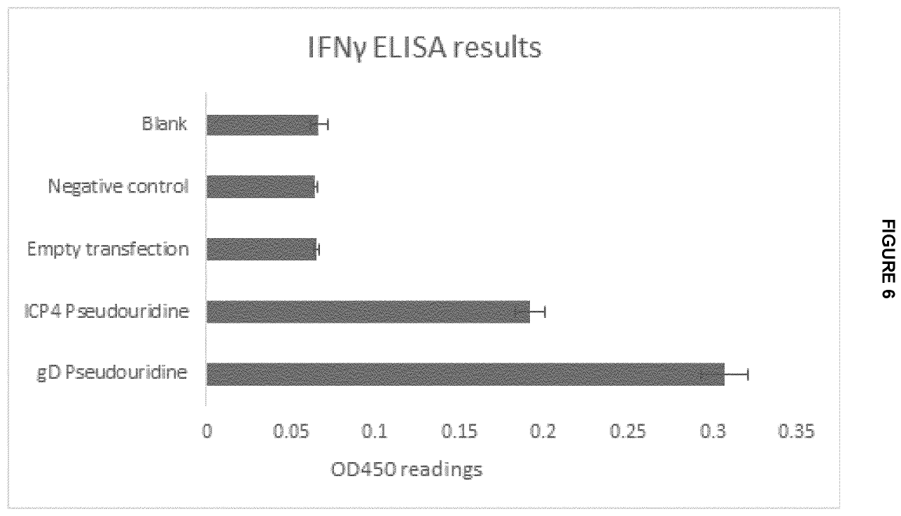

FIG. 6 shows a measurement of IFNγ release using ELISA upon incubation with the ICP4 and gD modified mRNAs, the empty transfection wells had only the transfection reagent added, nothing was added to the negative control wells. For the blank wells no biological sample was added during the ELISA.

Conclusion:

The Western blot and PBMCs experiments were performed to assess the stability and functionality of the present mRNA constructs. The design of mRNAs to be used in a vaccine composition is crucial and therefore has been evaluated. With regards to stability, the present vaccine mRNAs comprise an optimized 5′ cap, 5′ and 3′ UTRs, and polyA tail. The Western blot analyses (FIGS. 3 and 4) clearly show a robust expression of the UL48, UL11, UL16, and UL21 mRNAs and indicate that the constructs are stable, which is fundamental for the efficacy of a vaccine comprised of said mRNAs.

In addition to the stability, it has also been confirmed that the immune responses can be triggered by the vaccine components. In that context, the vaccine mRNAs were optimized using modified residues with 1-methyl-pseudouridine to reduce the innate non-specific immune responses. The IFNγ ELISA results indeed indicate the specific release of this immune factor upon the incubation with the functional UL48, gD and ICP4 mRNAs (FIGS. 5 and 6).

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes.

Other embodiments are within the following claims. In addition, where features or aspects of the invention are described in terms of Markush groups, those skilled in the art will recognize that the invention is also thereby described in terms of any individual member or subgroup of members of the Markush group.

1. A vaccine composition comprising one or more mRNAs, wherein each of said mRNAs encodes a Herpes Simplex Virus (HSV) structural protein or an immunogenic fragment thereof selected from the group consisting of:
(i) UL48;
(ii) UL48 and UL49;
(iii) UL11, UL16 and UL21; or
(iv) UL31 and UL34.

2. The vaccine composition of claim 1, wherein UL48 has an amino acid sequence which is 80% or more identical to the amino acid sequence of SEQ ID NO: 6, UL49 has an amino acid sequence which is 62% or more identical to the amino acid sequence of SEQ ID NO: 7, UL11 has an amino acid sequence which is 75% or more identical to the amino acid sequence of SEQ ID NO: 1, UL16 has an amino acid sequence which is 72% or more identical to the amino acid sequence of SEQ ID NO: 2, UL21 has an amino acid sequence which is 80% or more identical to the amino acid sequence of SEQ ID NO:3, UL31 has an amino acid sequence which is 85% or more identical to the amino acid sequence of SEQ ID NO: 8, and UL34 has an amino acid sequence which is 70% or more identical to the amino acid sequence of SEQ ID NO: 8.

3. The vaccine composition of claim 1 or 2, wherein each of the said HSV mRNAs is capable of eliciting an immune response when administered in the form of a vaccine composition to a subject.

4. The vaccine composition of any one of claims 1 to 3, further comprising at least one mRNA encoding a Herpes Simplex Virus (HSV) glycoprotein selected from the group consisting of a) an HSV glycoprotein D (gD) or an immunogenic fragment thereof having an amino acid sequence which is 70% or more identical to the amino acid sequence of SEQ ID NO:11, b) an HSV glycoprotein B (gB) or an immunogenic fragment thereof having an amino acid sequence which is 70% or more identical to the amino acid sequence of SEQ ID NO:10, and c) an HSV glycoprotein E (gE) or an immunogenic fragment thereof having an amino acid sequence which is 70% or more identical to the amino acid sequence of SEQ ID NO: 4 or 80% or more identical to the amino acid sequence of SEQ ID NO: 5, or any combination thereof.

5. The vaccine composition of claim 4, comprising:
(i) UL48 and gD and/or gB, optionally ICP4;
(ii) UL 48 and UL49 with gE;
(iii) UL11, UL16, and UL21 with gE, gD, and/or gB; or
(iv) UL31 and UL34 with gD and/or gB.

6. The vaccine composition of claim 4 or 5, wherein said least one mRNA encoding a Herpes Simplex Virus (HSV) glycoprotein is a nucleoside modified mRNA comprising one or more pseudouridine residues, preferably where the one or more pseudouridine residues comprise m¹ψ(1-methylpseudouridine); m¹acp³ψ(1-methyl-3-(3-amino-5-carboxypropyl)pseudouridine), ψm (2′-0-methylpseudouridine), m⁵D (5-methyldihydrouridine), m³ψ(3-methylpseudouridine), or any combination thereof.

7. The vaccine composition of any one of the preceding claims, wherein said at least one mRNA encodes HSV-1 polypeptides.

8. The vaccine composition of any one of the preceding claims, wherein said at least one mRNA encodes HSV-2 polypeptides.

9. The vaccine composition of claim 6, wherein said nucleoside modified mRNA encoding said immunogenic fragment of
(i) HSV gD comprises amino acids 26-331 from HSV-2 strain 333, or a homologous sequence from another HSV strain, preferably wherein the nucleic acid sequence of said nucleoside modified mRNA is as set forth in SEQ ID NO: 12; and/or
(ii) HSV gE comprises amino acids 24-405 from HSV-2 strain 2.12, or a homologous sequence from another HSV strain, preferably wherein the nucleic acid sequence of said nucleoside modified mRNA is as set forth in SEQ ID NO: 13.

10. The vaccine composition of any one of claims 1-9, wherein one or more of said mRNAs further comprises;
(i) a poly-A tail, preferably according to one or more of SEQ ID NOs: 25-30;
(ii) an m7GpppG cap, 3′-0-methyl-m7GpppG cap, or anti-reverse cap analog;
(iii) a cap-independent translational enhancer; and/or
(iv) 5′ and 3′ untranslated regions that enhance translation, preferably according to one or more of SEQ ID NOs: 25-30.

11. The vaccine composition of any one of claims 1-10 wherein one or more of said mRNAs are encapsulated in a nanoparticle, lipid, polymer, cholesterol, or cell penetrating peptide, preferably in a liposomal nanoparticle.

12. The vaccine composition of any one of claims 1 to 11 for use in treating or preventing a Herpes Simplex Virus (HSV) infection in a subject.

13. The vaccine composition for the use of claim 12, wherein said HSV infection is selected from the group consisting of an HSV-1 infection, an HSV-2 infection, a primary HSV infection, a flare, recurrence, or HSV labialis following a primary HSV infection, a reactivation of a latent HSV infection, an HSV encephalitis, an HSV neonatal infection, a genital HSV infection, or an oral HSV infection.

14. The vaccine composition for the use of claim 13 or 14, wherein the vaccine composition is for intramuscular administration, subcutaneous administration, intradermal administration, intranasal, intravaginal, intrarectal administration, or topical administration, preferably wherein the composition is a vaccine for injection.

15. The vaccine composition of any one of the preceding claims, further comprising a pharmaceutically acceptable carrier or adjuvant for injection.
